(19) United States
(12) Patent Application Publication
(10) Pub. No.: US 2012/0213826 A1
(43) Pub. Date: Aug. 23, 2012

(54) Use of Pro-Inflammatory Compounds for Promoting Bone Formation

(75) Inventors: Jagdeep Nanchahal, London (GB); Nicole Horwood, London (GB)

(73) Assignee: Imperial Innovations Limited, London (GB)

(21) Appl. No.: 13/383,640

(22) PCT Filed: Jul. 13, 2010
(86) PCT No.: PCT/GB10/01340

§ 371 Date: May 10, 2012

(30) Foreign Application Priority Data
Jul. 14, 2009 (GB) 0912159.1

(51) Int. Cl. A61K 38/19; A61P 19/08; A61P 19/10; A61K 9/00; A61P 19/00

(52) U.S. Cl. 424/400; 424/85.1

(57) ABSTRACT

The present invention provides methods, uses and compounds for promoting bone formation in a patient at a site in need thereof by the provision of a pro-inflammatory compound at the site. The site is generally a site of injury or a site of surgical intervention in the patient. Exemplary compounds include pro-inflammatory: cytokines. Inhibitors of indoleamine 2, 3, dioxygenase 1 are also provided in the methods and uses of the invention.

The present invention relates to methods and uses in relation to accelerating bone formation and the quality of the bone in individuals, including those with fractures and damaged bones, and those requiring implant fixation and fusions.

Approximately 3% of the population per annum sustain a fracture and up 40% of these involve high-energy trauma. Incidence of fractures in adults in the USA: 2,800 per 100,000 person years (2.8%): 2,200 due to moderate trauma, 1,200 (1.2%) due to severe trauma and 200 pathological fractures. In Edinburgh, for a population of 600,000, 16,000 adults sustained fractures over a 1 year period (2.7%).

The tibia is the most commonly fractured long bone. High-energy fractures of the tibial shaft are limb-threatening injuries. High-energy tibial fractures take on average 42 weeks to heal and 13% develop a non-union (Bosse et al. (2002) N Engl J Med. 347(24): 1924-31).

Currently, the only biological therapy for stimulating fracture healing involves the introduction of bone morphogenetic proteins (BMPs), although clinical trials of BMPs 2 and 7 failed to replicate the efficacy achieved in animal models (Lieberman J R, et al (2002) J Bone Joint Surg Am. 84-A(6): 1032-44). This may reflect the failure of a single supra-physiological dose to replicate the complex cascade of growth factor production seen in vivo. Hence, the clinical benefit of BMPs remains unrealised and an alternative approach is required.

Since it was shown that bone growth factors (BMPs) present in demineralised matrix could induce bone formation in muscle (Urist M R. (1965) Science. 150(698): 893-9), the concept of using muscle derived stromal cells (MDSC) to aid fracture healing has been investigated (Lieberman J R, et al (2002) supra). However, reintroduction following ex vivo expansion of MDSC (virally transduced to express BMPs) (Musgrave D S, et al. (2002) J Bone Joint Surg Br. 84(1): 120-7; Shen H C, et al. (2004) J Gene Med. 6(9): 984-91; and Wright V, et al. (2002) Mol Ther, 6(2): 169-78) is time consuming and has translational complications.

Inflammation plays a vital role in early fracture repair. In murine models, TNF-α, IL-1 and IL-6 are expressed at the fracture site within 24 hrs following injury (Kon T, et al. (2001) J. Bone Miner. Res. 16(6): 1004-14; Cho T J, et al (2002) J Bone Miner Res. 17(3):513-20). TNFα and IL-1 follow a biphasic pattern. Early synthesis by macrophages and other inflammatory cells induces the release of secondary signalling molecules, some of which mediate osteoprogenitor chemotaxis and differentiation (Kon et al., 2001, supra). The later peak co-ordinates the transition from chondrogenesis to osteogenesis during endochondral maturation (Kon et al, 2001, supra; Lehmann W, et al. (2005) Bone. 36(2): 300-10). A closed tibial fracture model using TNFα receptor (p55^{-/-}/p75^{-/-}) knockout mice demonstrated delayed chondrogenesis and endochondral maturation (Gerstenfeld L C, at al. (2003) J Bone Miner Res. 18(9): 1584-92). IL-6, produced by osteoprogenitors in response to TNFα and IL-1, stimulates osteoBIastic differentiation and elicits an anti-apoptotic effect (Heymann D & Rousselle A V. (2000) Cytokine. 12(10): 1455-68). A femoral fracture model using IL-6 knockout mice demonstrated delayed callus remodelling and mineralization (Yang X, at al. (2007) Bone. 41(6): 928-36). Both IL-1β and TNFα have been shown to stimulate proliferation of osteoBIasts, whilst IL-6 had no effect (Frost A, at al. (1997) Acta Orthop Scand. 68(2): 91-6). IL-1β and TNFα have also been shown to recruit osteoprogenitors by inducing RANTES (CCL5) secretion that acts in an autocrine and paracrine fashion (Yano S, et al. (2005) Endocrinology. 146(5): 2324-35).

Therefore, the pro-inflammatory environment has been implicated in the recruitment, proliferation and differentiation of osteoprogenitors and may be involved in the early stages of endochondral fracture healing. However, there have been conflicting reports in this area and some studies have suggested that an exaggerated inflammatory response actually delays healing of fractures.

Open fractures (such as high-energy fractures as discussed above) are slow to heal for reasons that are unknown. Some studies have implicated a prolonged inflammatory response in open fractures as a factor that contributes to and perhaps even causes the slow healing that is seen.

Bunn et al (2004) J Orthop Res 22: 1336-44 observed that poor healing of high-energy fractures is often associated with severe muscle crush. In a rat model of muscle crush, they found that in the large crush group there was significantly higher expression of IL-1β, IL-6 and TNFα as well as greater inflammatory cell infiltrate. They concluded that increased production of inflammatory cytokines such as TNFα and IL-1β may lead to delayed or non-union of fractures.

Kratzel et al (2008) BMC Musculoskeletal Disorders 9: 135—also suggests that delayed fracture healing (in open tibial osteotomies) was due to prolonged inflammation. The authors concluded that the most plausiBIe explanation for non-unions [of fractures] was aseptic inflammation. The authors suggest that “regardless of the importance of inflammation for initialising the healing process, severe soft tissue trauma and the linked excessive release of inflammatory mediators can be discussed as factors to have a negative impact on bone healing”.

Hashimoto et al, (1989) Bone. 10: 453-457 describes a study on the effect of Tumour Necrosis Factor (TNFα) administration on healing of fractured ribs of rats. They found that fracture healing was significantly inhibited by daily intraperitoneal administration of recombinant human TNFα (400 μg/kg body weight per day) after fracture. Histological examination showed that TNFα inhibited cartilagenous callus formation. They concluded that TNFα inhibits cartilage formation in the early phase of bone induction in fracture healing and suggested that this effect of TNFα was due to its inhibition of differentiation of mesenchymal cells into chondroBIasts.

Lacey D. C. et al (2009) Osteoarthritis and Cartilage 17: 735-42 showed that osteogenic differentiation from a population of mesenchymal stem cells was suppressed by IL-1β and TNFα. Therefore, Lacey et al, suggests that TNFα inhibits bone formation by mesenchymal stromal cells and hence may adversely affect bone healing.

Further, multiple studies have shown that bone erosions in patients with rheumatoid arthritis can be healed with administration of anti-TNFα antibodies during the early phases of the disease (Feldmann M and Maini R (2001) Ann Rev Immuno) 19:163-196; Hirose et al. (2009) Mod Rheumatol 19(1):20-26). Furthermore, inflammation is central to aseptic implant loosening and inhibition of TNFα in animal models has been shown to reduce osteolysis (Purdue P E et al, 2006, HSS J 2: 102-113). Thus, the skilled person would consider that an upregulated inflammatory response is inhibitory of bone formation and fracture healing and would expect such an upregulation to be undesiraBIe in patients with broken, damaged or eroded bones.

Further studies have investigated the role of TNFα in the processes involved in fracture healing.

Kon, T. et al (2001) J Bone Mineral Res. 16: 1004-14 followed the healing of closed tibial fractures in mice. TNFα and IL-1 are expressed at both very early and late phases in the repair process, which suggests that these cytokines are important in the initiation of the repair process and play important functional roles in intra membranous bone formation and trabecular bone remodelling. Kon et al suggests that IL-1 participates in osteoBIast proliferation and differentiation. However, it is not clear whether this is a direct effect of IL-1 or if it is mediated through actions of TNFα. IL-1α and TNFα are synthesised not only by macrophages and inflammatory cells recruited to the site of injury but also by mesenchymal cells in the periosteum. TNFα also appears to be synthesised by hypertrophic chondrocytes and both IL-1 and TNFα are synthesised by lining cells on the newly formed trabecular bone surfaces. Kon et al suggest that TNFα may potentially to regulate the initiation of fracture healing including mesenchymal cell proliferation and differentiation in the periosteum, although no definite data were provided.

Gerstenfeld et al (2003) J Bone Mineral Res. 18: 1584-92 studied the healing of closed tibial fractures in p55^{-/-}/p75^{-/-} mice (TNFα receptor knockout mice). Chondrogenic differentiation was delayed by 2-4 days but subsequently proceeded at an elevated rate. Endochondral resorption was delayed by 2-3 weeks. This was in line with results obtained in an earlier study by Gerstenfeld et al (2001; Cells Tissues Organs 169: 285-94).

Gerstenfeld (2003) J Bone Mineral Res. 18: 1584-92 concluded that TNFα participates at several functional levels, including the recruitment of mesenchymal stem cells, apoptosis of hypertrophic chondrocytes and the recruitment of osteoclasts. Repair of injured bone required the co-ordinated participation of haematopoietic and immune cell types within marrow space in conjunction with the vascular and skeletal cell precursors recruited from the periosteum and surrounding soft tissues. Gene expression studies for extracellular matrix products associated with chondrogenic differentiation suggested that differentiation of mesenchymal cells to chondrocytes was delayed in TNFα receptor knockout mice. However, once chondrogenic differentiation was initiated, enhanced expression of these genes was observed and differentiation proceeded at the normal rate. TNFα mediates chondrocyte apoptosis and regulates endochondral tissue resorption (Gerstenfeld (2003)). Gerstenfeld (2003) set out to determine the functional role of TNFα in closed fracture healing after BIunt trauma. Three effects of the absence of TNFα signalling were seen in the experiments conducted: (1) a delay during the initial healing phase in either recruitment of mesenchymal cells or their osteogenic differentiation, (2) a delay in chondrocyte apoptosis during the endochondral period, and (3) a delay in resorption of mineralised cartilage during the endochrondral period.

Gerstenfeld (2003) also concluded that whilst the current paradigm is that endochondral progression during post natal fracture repair recapitulates the processes that occur during embryological skeletal development, current data suggests this is not the case as TNF p55^{-/-}/p75^{-/-} animals show no overt skeletal abnormalities. Thus, fracture repair has numerous complexities that are distinct from embryological skeletal tissue formation and may not be regulated by the same mechanisms.

Lehmann (2005) Bone. 36: 300-310 suggested that TNFα signalling in chondrocytes controls vascularisation through the regulation of angiopoietin and vascular endothelial growth inhibitor. TNFα also in part regulates MMP9 and MMP14, which are known to be crucial to the progression of vascularisation and turnover of mineralised cartilage. Therefore, TNFα signalling in healing fractures co-ordinates the expression of specific regulators of endothelial cell survival and MMPs and is essential in the transition and progression of the endochondral phase of fracture repair.

Mountziaris, P. M. and Mikos, A. G. (2008) Tissue Engineering Part a 14(2): 179-describes studies that indicate that proinflammatory cytokines may be important for triggering tissue regeneration following injury. This review hypothesised that rational control of inflammation should be incorporated into the design of tissue engineering strategies.

Fractures heal by a combination of intramembranous and endochondral ossification. Intramembranous bone formation occurs beneath the periosteum and results in the formation of hard callus, where bone is formed directly, with no intermediate cartilage stage. Bone formed by endochondral ossification involves a cartilage intermediate.

Gerestenfeld et al (2001) Cells Tissues Organs, 169: 285-294, studying fracture healing in TNFα receptor deficient (p55^{-/-}/p75^{-/-}) mice using a model which differentiated intramembranous and endochondral healing suggested that a different set of signals are involved during endochondral and intramembranous bone formation. They found a complete absence of bone formation on the periosteal surface whilst although initially there was a delay in endochondral healing, at later time points the processes were accelerated. They concluded that TNFα plays a crucial role in the post natal period during intramembranous bone formation in fracture healing whilst different signals may be involved in endochondral bone formation.

The present invention is based on (though not restricted to the application of) the surprising discovery that a pro-inflammatory response in a mouse model of severe fractures (where the bone had been stripped of periosteum to mimic high-energy fracture) led to a significant reduction in the time taken for the fractures to heal. The inventors surprisingly found that the administration of pro-inflammatory cytokines led to accelerated healing compared with that seen in control mice. This is in contrast to the many puBIications suggesting proinflammatory cytokines delay fracture healing. Further, the inventors identified indoleamine 2, 3, dioxygenase 1 (IDO) as a key regulator in the inflammatory response that led to healing of bone following fracture in the mouse model. The inventors unexpectedly found that the use of an IDO inhibitor or the knockout of the

IDO gene in mice led to a significant reduction in the time taken for a fractured bone to heal. This surprisingly resulted in a dramatic reduction in the time taken for the bone to heal and consolidate. The inventors considered that the absence of IDO activity would lead to an uncontrolled inflammatory response that would have inhibited healing or further damaged the injured area. Surprisingly, this did not happen.

Thus, in a first aspect, the present invention provides a method of promoting bone formation in a patient at a site in need thereof, the method comprising the step of administering a pro-inflammatory compound to the site.

A second aspect of the invention provides the use of a pro-inflammatory compound in the manufacture of a medicament for promoting bone formation in a patient at a site in need thereof, wherein the medicament is for administering the compound to the site.

In a third aspect, the present invention provides a pro-inflammatory compound for use in promoting bone formation in a patient at a site in need thereof. In a preferred embodiment the pro-inflammatory compound is for administering to the site in the patient.

The site in need of the promotion of bone formation may be any number of areas comprising bone that is injured, damaged, eroded, brittle, or defective in some other way such that it would benefit from the promotion of bone growth at that site. The promotion of bone growth is envisaged to lead to the acceleration of bone growth at that site in comparison with the rate of bone growth seen in patients who are not subject to the present invention.

Thus, the site may be a site of injury. Alternatively, the site may be a site of surgical intervention.

By “site of an injury” we include the meaning that the site may be the site of an injury, such as the fracture of a bone. By “site of surgical intervention” we include the meaning that the site may be a site of a surgical intervention, such as the insertion of an implant into a bone. The site may also be a combination of both a site of injury and a site of surgical invention. In other words, when the site is one of both an injury and a surgical intervention, this may be, for example, the placing of an implant at the site of a fracture. Alternative embodiments of such sites that fall within the intended scope of the present invention will be immediately apparent to a person of skill in the art.

The site may be a site requiring bone fusion or comprising damaged bone, eroded bone or bone defects. Such embodiments may also be found in combination with each other or with a site of injury or surgical intervention.

It is envisaged that a site where there is damaged and/or eroded bone may be more prone to injury, such as fragility fractures experienced by sufferers of conditions such as osteoporosis. Further, in patients where a site requires bone fusion, one may expect that that site may also be a site of injury, which injury may have led to the requirement for the fusion of a bone. For example, a spinal injury may call for the fusion of two vertebrae to stabilise the spine. Alternatively, it may be a site of other pathology, for example due to degeneration between vertebrae resulting in the site being treated by surgical fusion of the vertebrae.

By “site comprising bone defects” we include the meaning that the bone at that site has a defective composition or structure in comparison to healthy bone. Such defects may be congenital or they may be acquired through injury or disease or other cases as would be well known to a person of skill in the art. That a site has “bone defects” or damaged bone may be assessed, for example, radiologically (e.g. by X-ray or by CT scan), as would be appreciated by a skilled person.

The present invention may be useful in repairing damaged and/or eroded bone. By “damaged and/or eroded bone” we include the meaning that the bone has accumulated damage though environmental factors or genetic factors that have left the bone in a state of fragility and where the bone is weak and prone to fracture. Thus, the present invention may be useful in repairing bone after osteomyelitis (infection of the bone) has damaged the bone. It is also envisaged that the present invention will be useful in repairing bone damage after irradiation or chemotherapy, in patients with bone metastases of tumours or multiple myeloma. Congenital and other defects of bone may also be repaired using the methods and uses of the present invention.

It is envisaged that in any aspect of the present invention, the promotion of bone formation will aid in healing the site. As indicated above, the site may be a site of injury and/or surgical intervention. It is expected that the site of injury or surgical intervention will comprise damaged or broken bone. The promotion of bone formation is considered therefore to aid in healing fractures or fissures in the bone and in improving the strength, flexibility and/or quality of the bone and in fusing bone and/or repairing bone, as appropriate.

Thus, in a preferred embodiment of the present invention the site may be a site of injury and the injury may be a fracture of a bone. The present invention is considered to be particularly useful in repairing bone that has been severely fractured in a high-energy impact with periosteal stripping, such as illustrated in FIG. 1 and comminution resulting in multiple fragments.

The present invention is also considered to be useful in repairing less severely damaged bone, thus allowing the tissue layers or bone that were present at the site before the injury or surgical intervention occurred to be replenished. Such an embodiment is considered to be particularly useful after the insertion of an implant into the bone, where new bone formation is considered to enaBIe the implant to adhere more securely than it would in the absence of the effects of the present invention. Examples include fixation of screws and implants for joint replacement.

The present invention may allow modulation of bone healing to accelerate as well as improve the quality of healing. This would allow for faster union and improved consolidation of the fracture or implant fixation. In the clinical scenario, there is a race between fracture union/consolidation and implant failure, especially in compromised bone as exemplified by fragility fractures. The invention is considered to promote union and consolidation, thereby reducing complications at the fracture or implant site and allow more rapid mobilisation of the patient.

In an embodiment of any aspect of the invention the surgical intervention may be an osteotomy. By “osteotomy” we include any surgical procedure where bone is purposefully cut to shorten, lengthen or otherwise change its alignment. The present invention is envisaged to provide a means by which the healing process, after such a procedure, may be accelerated.

In certain surgical procedures, bone grafts are utilised to accelerate growth and healing of bone. With the application of the present invention in addition to a bone graft, it is envisaged that the bone healing process may be accelerated further. An example of when a bone graft may be used includes instances when the fusion of bone is required. It is considered that the present invention will not only aid in the acceleration of the healing of a site of grafted bone but also in healing the site where the donor bone has been excised. Thus, in an embodiment the surgical intervention may be the removal of bone from a donor site for a bone graft. In a further embodiment the site of surgical intervention may be the site of a bone graft itself. It is considered that the promotion of bone formation will aid in repairing bone at the site and/or accelerating bone formation at the site.

In an embodiment of any aspect of the present invention it is considered that the promotion of bone formation at the site may aid in repairing bone at the site and/or accelerating bone formation at the site and/or increasing cortical bone volume and/or cortical bone mineral content and/or bone mineral density at the site and/or increasing mineralised volume of the healing bone and/or the mineralised bone volume fraction and/or tissue mineral density at the site. Thus, the invention may increase the bone mineral density and/or bone volume and/or mature bone content at the site. It is further considered that the present invention may lead to an improvement in the stiffness of the bone at a fracture site. Thus, the present invention may aid in improving the strength of the newly formed bone and may reduce the likelihood of further fractures or other pathologies at that site.

In an alternative embodiment, the surgical intervention may be a procedure for inserting an implant into, around and/or adjacent to a bone. Alternatively, the surgical intervention may be for fixing an implant to a bone. Such implants may be selected from, but not limited to, the group comprising a joint replacement, a dental implant, a pin, a plate, a screw, an intradmedullary or intraosseous device. Exemplary joint replacements include hip replacements, knee replacements, shoulder replacements and elbow replacements. Dental implants may include implants into the mandiBIe or maxilla to support crowns or other prosthetic dental structures or other implants as would be appreciated by a person of skill in the art. Further joints that may receive implants include the ankles, wrists, digits and spine. Pins and plates may be inserted to strengthen a bone or joint after an injury, such as a fracture. Promoting fixation may also be useful in osteointegrated implants, e.g. teeth, digits, facial prosthesis and hearing devices.

Current literature would suggest that the majority of implant failures in joint replacements occur as a result of loosening. Loosening can be due to infection or aseptic. In both instances, excessive inflammation is universally acknowledged to be involved in the underlying mechanism (See Dempsey et al (2007) Arthritis Research & Therapy 9: R46; Li et al (2009) BMC Musculoskeletal Disorders 10: 57; Cheng & Zhang (2008) Medical Hypotheses 71: 727-29; and Purdue et al (2006) HSSJ 2: 102-13).

Intradmedullary implants are currently either inserted with cement or they are inserted uncemented/cementless. The latter have characteristics which encourage bone formation into the intraosseous component by a variety of techniques, including surface coatings such as hydroxyapatite or the presence of biocompatiBIe material and surface characteristics which encourage bone formation and ingrowth. Biological fixation of cementless implants, as exemplified by acetabular cups in hip arthroplasty, requires initial implant stability and physical interlocking between the cup and the supporting bone to allow bone ingrowth. This is crucial for long term stability, although initial stability can be provided from a porous-coated surface or an adjunct fixation with spikes or screws, there are potential proBIems with these methods:

1 Coatings can cause proBIems including de-bonding and bead shedding

2 Spikes or screws are associated with alterations in load distribution and local bone damage during implant insertion.

Rapid ingrowth onto the acetabulum cup by a biological method of enhanced bone formation, as in the present invention, would reduce the need for adjunct fixation, expensive implant coatings and would simplify surgical technique.

Thus, it is considered that the present invention may improve adherence of the implant to the bone. By “improve adherence” we include the meaning that the adherence of the implant to the bone would be stronger in a patient who is subject to the present invention over a patient who is not. The adherence may also be more efficient and effective adherence of the implant to the bone may be achieved at an accelerated rate (i.e. occur more rapidly) compared with adherence of an implant in the absence of the methods, uses and compounds of the present invention.

Thus in an embodiment of the preceding aspect, the adherence of the implant to the bone is strengthened in comparison with adherence of an implant to bone in the absence of the present invention. The improvement in adherence may occur through newly formed bone fusing with the implant and securing the implant into place. Alternatively, or additionally, the present invention may improve the strength of the bone that is adjacent to the implant site and thus structurally improve and strengthen the area of bone housing io the implant. The improved adherence of the implant to the site is envisaged to be of particular benefit where the implant is intended to remain at the site of surgical intervention for an extended period, or permanently.

The skilled person would appreciate the standard techniques in the art that are used to assess the strength and efficiency of adherence of an implant to the bone at the implant site. For example, the adherence of the implant may be measured using radiological assessment of the site. This provides for the measurement of the peri-implant bone quality and allows the physician to quantify the success of the implant. Assessment of implant adherence and the quality of the fixture may also be assessed over the longer term by assessing the stability and longevity of the implant at the site. If the fixture of the implant loosens, then the patient will generally report pain and other symptoms. Aseptic loosening accompanied by periprosthetic osteolysis is one of the leading complications of joint replacement. Thus, the present invention may reduce complications of implant surgery and improve patient quality of life. For implants used to stabilise fractures, accelerated and improved bone healing will permit faster mobilisation of the patient and reduced incidence of implant failure.

Thus, the implant may have a reduced tendency to loosening from the site of insertion in comparison with an implant inserted in the absence of the present invention. This would be assessed by assessing patient symptoms (including pain) and assessing the patient radiologically and/or mechanically, as would be appreciated by a skilled person.

In an embodiment of the invention, the fractured bone has a disrupted or damaged periosteum and/or endosteum. By “disrupted or damaged periosteum and/or endosteum” we include the meaning that the periosteal and/or endosteal membranes that surround the bone have been broken, damaged or even completely removed such that they are no longer attached to the bone or no longer cover or envelope the bone. A common cause of a fracture resulting in a disrupted or severely traumatised periosteum and/or endosteum is a high-energy fracture. By “high-energy fracture” we include the meaning that the bone has been fractured in a high-energy collision, where a large force has collided with the bone, transferred a large amount of energy, and caused severe trauma. This commonly occurs during road-side accidents involving vehicles colliding with pedestrians, cyclists or motorcyclists at high speed. For this reason, such high-energy fractures commonly involve the bones of the lower leg, in particular the tibia. The present invention is envisaged to be useful in aiding in the healing of high-energy fractures. The endosteum is specifically removed during reaming of the medullary canal for insertion of implants with an intramedullary component.

The invention is also considered to be useful in aiding in the healing of less severe fractures and minor fractures. The types of fracture where new bone formation may be beneficial to the patient will be understood by a person of skill in the art. Thus, the present invention will be expected to be beneficial in non-periosteally stripped fractures as well as periosteally stripped fractures (see Gerstenfeld et al, (2001) Cells Tissues Organs 169: 285-294,).

Thus, it is considered that the present invention will also be beneficial in patients who have a fracture where the fractured bone has an intact periosteum and/or endosteum.

Such fractures may include “closed” fractures where the soft tissue envelope has not been broken or disrupted by the impact that led to the injury. The present invention may be used in such circumstances to significantly improve and accelerate healing of the fractured bone through promoting the formation of new bone.

It is considered that the promotion of the formation of new bone by the methods, uses or compounds of the invention may be particularly useful in patients who have weakened or more brittle bone in comparison with what would be considered healthy bone. For example, such patients may have osteoporosis. Other examples of conditions or pathologies where patients may have weakened or more brittle bones and who may benefit from the methods and uses of the present invention include those who have compromised bone due to metabolic bone disorders, hereditary bone conditions, infection, malignant or benign tumours affecting bone, bone affected by chemotherapy, radiotherapy and/or disuse.

Thus, the patient may have compromised bone due to metabolic bone disorders hereditary bone conditions, osteoporosis, infection, malignant or benign tumours affecting bone, bone affected by chemotherapy, radiotherapy and/or disuse.

For example, patients with osteoporosis have reduced bone mineral density (BMD), the bone microarchitecture is disrupted, and the amount and variety of non-collagenous proteins in the bone is altered. This leads to bones that are more likely to experience fractures than healthy bone. The present invention may be used to increase the strength and density of bone in such patients whether they have undergone surgery or have io suffered a fracture.

Thus, the present invention may lead to newly formed bone that has improved bone quality, quantity, density and shorter healing times in patients with fragility fractures in comparison with the compromised bone that was previously present at the site of injury or surgical intervention in such patients. Improved healing, gauged in terms of increased bone quality, strength, density and/or quantity could be assessed clinically, as well as by radiological techniques, including quantitative CT scanning and/or dual energy x-ray absorbiometry (DXA). DXA measures bone mineral density and may be used to quantify this variaBIe. The present invention may also be beneficial in patients who have other bone weakening conditions such as rheumatoid arthritis, dental caries, osteomyelitis, tumour metastases in bone and multiple myeloma or radiotherapy or chemotherapy.

In an alternative embodiment, the present invention may be useful in promoting bone formation in situations where the fusion of two or more bones is required. For example, where a joint is weak or unstaBIe, it may be desiraBIe to fuse the bones at the joint to increase stability. For example, bone fusion may be required at the vertebrae. Where a vertebrae is damaged, fusion of the damaged vertebrae to the adjacent vertebrae may improve stability of the spine. Thus, the present invention may be used to aid in the fusion of bone. Typically, in order to carry out a bone fusion, a graft of bone from another site on the patient's skeleton is taken and is transplanted at the site of desired bone fusion in order to promote bone fusion at the site of weakness. Such a bone graft may be taken, for example, from the pelvis. The bone graft (autogenous or otherwise) will typically be aided with the use of bone conducting and induction substances such as inorganic and organic matrices. Such bone induction substances may include bone morphogenic proteins.

It is considered that the present invention is not only beneficial in promoting bone formation at the site where bone fusion is encouraged, and thus accelerating the process of bone fusion, but the present invention is also useful in promoting bone formation at the site from where the bone sample is taken, thus accelerating healing at the donor site as well.

Thus, the present invention will aid in bone fusion (e.g. joint fusion) and it is considered that the methods, uses and compounds of the present invention will replace or augment the addition of a bone graft (autogenous or otherwise), and bone conducting and/or induction substances.

In a further embodiment, the present invention may be used to address bone defects. Examples of situations that may lead to bone defects include comminution at a fracture site and bone loss (such as through fracture fragmentation, which can be segmental or periprosthetic).

In a further embodiment, the present invention may be used to augment and accelerate bone formation during distraction lengthening. Distraction lengthening may occur in, for example, the mandiBIe and long bones.

In a further embodiment, the present invention may be used to accelerate bone formation in tissue engineered constructs. Attempts to engineer bone, either in the body or outside it, have met with limited success. The constructs have not found widespread clinical application due to the slow formation of bone capaBIe of bearing load. The present invention may be used to accelerate bone formation and maturation of that bone in tissue engineered constructs.

In an embodiment of all aspects of the present invention, the compound may be administered (in the methods of the invention), or may be for administration (in the uses and compounds of the invention) directly to the site. Alternatively, in certain embodiments, it may be appropriate to administer the compound of the invention systemically. An example of an embodiment where systemic administration may be appropriate includes administration to patients with vertebral fragility fractures.

It is envisaged that the compound may be formulated as appropriate for the type of injury or surgical intervention in question. Appropriate formulations will be evident to a person of skill in the art and may include, but are not limited to, the group comprising a liquid for injection or otherwise, an infusion, a cream, a lozenge, a gel, a lotion or a paste. The compounds of the invention may also be for administration in biocompatiBIe organic or inorganic matrices including, but not limited to, collagen or fibronectin matrices. It is envisaged that such matrices may act as carriers of the compound in an appropriate formulation or may aid in the promotion of bone formation by augmenting the effects of the compound.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

In human or animal therapy, the compounds of the invention can be administered alone but will generally be administered in admixture with a suitaBIe pharmaceutical excipient diluent or carrier selected with regard to the intended route of administration and standard pharmaceutical practice.

The compounds of the invention can be administered parenterally, for example, intravenously, intra-arterially, intraperitoneally, intrathecally, intraventricularly, intrasternally, intracranially, intra-muscularly or subcutaneously, or they may be administered by infusion techniques. They may be best used in the form of a sterile aqueous solution which may contain other substances, for example, enough salts or glucose to make the solution isotonic with BIood. The aqueous solutions should be suitaBIy buffered (preferaBIy to a pH of from 3 to 9), if necessary. The preparation of suitaBIe parenteral formulations under sterile conditions is readily accomplished by standard pharmaceutical techniques well-known to those skilled in the art.

Formulations suitaBIe for parenteral administration include aqueous and non-aqueous sterile injection solutions which may contain anti-oxidants, buffers, bacteriostats and solutes which render the formulation isotonic with the BIood of the intended recipient; and aqueous and non-aqueous sterile suspensions which may include suspending agents and thickening agents. The IDO inhibitor 1-methyl-d-tryptophan or its derivatives may be formulated, for example, as described in Taher et al. (2008) J. Allergy Clin. Immunol. 121(4): 983-91. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and taBIets of the kind previously described.

Alternative IDO inhibitors may also be used, as could therapeutic forms of pro-inflammatory cytokines (discussed further below) such as TNFα and IL-1β, including but not limited to long-lived forms. The half-life of proinflammatory cytokines such as TNFα and IL-1β could be increased by coupling to carrier proteins such as serum albumin and IgGFc.

Preferred unit dosage formulations are those containing a daily dose or unit, daily sub-dose or an appropriate fraction thereof, of an active ingredient. It is preferred that doses for topical administration of 1-methyl tryptophan may be of the order of fractions of or multiple mg/kg body weight of the patient. For example, the dose may be between 0.01 to 500 mg/kg body weight; 1 to 400 mg/kg body weight; 2 to 200 mg/kg body weight; 3 to 100 mg/kg body weight or 4 to 50 mg/kg (or any combination of these upper and lower limits, as would be appreciated by the skilled person). The dose used may in practice be limited by the solubility of the compound. Examples of possiBIe doses are 0.01, 0.05, 0.075, 0.1, 0.2, 0.5, 0.7, 1, 2, 5, 10, 12, 15, 20, 25, 30, 35, 40, 45, 50 or 100 mg per kg body weight up to, for example 500 mg/kg body weight, or any value in between. It is envisaged that preferred doses of other IDO inhibitors would be adjusted according to relative potency. The physician or veterinary practitioner will be aBIe to determine the required dose in a given situation based on the teaching and Examples provided herein. For example, doses may be determined using techniques as described herein, for example in the Examples. For example, the in vitro system used in FIG. 10 may be used to determine relative potencies of different candidate compounds. This information can then be used to determine an initial dose for testing in a dose-escalation manner in appropriate patients, as well known to those skilled in the art.

Alternatively, the compounds of the invention may be applied topically in the form of a lotion, solution, cream, ointment or dusting powder. The compounds of the invention may also be transdermally administered, for example, by the use of a skin patch.

For application topically to the skin, the compounds of the invention can be formulated as a suitaBIe ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitaBIe lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol, water and dimethyl sulphoxide (DMSO).

Formulations suitaBIe for topical administration in the mouth (such as in the dental embodiments of the invention) include lozenges comprising the active ingredient in a flavoured basis, usually sucrose and acacia or tragacanth; pastilles comprising the active io ingredient in an inert basis such as gelatin and glycerin, or sucrose and acacia; and mouth-washes comprising the active ingredient in a suitaBIe liquid carrier.

It should be understood that in addition to the ingredients particularly mentioned above the formulations of this invention may include other agents conventional in the art having regard to the type of formulation in question, for example those suitaBIe for oral administration may include flavouring agents.

For veterinary use, a compound of the invention is administered as a suitaBIy acceptaBIe formulation in accordance with normal veterinary practice and the veterinary surgeon will determine the dosing regimen and route of administration which will be most appropriate for a particular animal.

Endochondral bone formation proceeds through a series of discrete stages; tissue damage and disruption of BIood supply are followed by haematoma formation within hours. Acute inflammation in response to traumatic injury likewise occurs within minutes/hours of the insult. Thus it will be important to enhance aspects of this phase of the response. The formation of a cartilage intermediary followed by new bone formation occurs in the subsequent days and weeks. Hence the requirement for TNFα at the cartilage remodelling phase is distinct from its role in the initial stages of fracture repair.

The invention provides for the promotion of fracture healing through the surprising discovery that initially promoting the inflammatory response accelerates the entire healing response. This is surprising because the plasma half life of TNFα is less than 1 hour, although this can be potentiated in the presence of soluBIe TNFα receptors up to 6 hours (Beutler et al. (1985) J. Immunol. 135(6):3972-3977; Aderka et al. (1998) J. Clin. Invest 101(3):650-659). Once stimulated using the invention, the entire process of fracture healing surprisingly proceeded at an accelerated rate,

Thus, in a further embodiment, the compound of the present invention may be administered (or be for administration) to the site, for example, immediately following injury or surgery. Alternatively, the compound may be administered (or be for administration) at any time after initiation of surgery or after injury, for example between one hour and one year after surgery or injury. Alternatively, this may be more than one year after surgery or injury. It is preferred that the compound is administered less than a day after surgery or injury, for example up to 30 minutes, 1 hour, 2, 3, 4 or 5 hours after the injury or surgical intervention. Alternatively, the compound may be administered or be for administration multiple days, weeks or even months after the injury or surgical intervention. The later time points for administration (i.e. weeks and months after the initial insult) may be relevant, for example, where a patient has a fracture and it is treated either with surgery or a plaster cast or not treated at all and the fracture does not unite. The compound may then be for administration several months later when the non-union is diagnosed. Alternatively, the compound may be for administration at the time of surgical intervention or injury.

A further aspect of the present invention provides a kit of parts comprising a surgical implant in combination with a pro-inflammatory compound. It is envisaged that the compound of the invention, in an appropriate formulation, may be applied topically with the implant, for example, as a paste or gel, a liquid or slow release formulation or in conjunction with organic and/or inorganic matrices. In an embodiment, the kit of the invention may further comprise cement suitaBIe for bonding the surgical implant to bone. The compound may be incorporated into the implant or into the bone cement or applied locally or administered subsequent to the placing of the implant, for example by injection.

When incorporated into the cement the compound may be dispersed within the cement or coated around the cement, as would be appreciated by a person of skill in the art. Cements that are suitaBIe for bonding the surgical implant to bone would be well known by a person of skill in the art. Currently availaBIe implant coatings include biocompatiBIe metals and hydroxyapatite. These coatings encourage bone ingrowth but suffer from complications including debonding of the coating.

It is envisaged that the compounds of the present invention will be used in place of, or in combination with, presently availaBIe coatings. Such combinations may lead to synergistic effects including improved implant adherence and reduced complications. It is considered that the compounds of the invention may gradually diffuse into the surrounding tissue and promote bone formation over time. It is considered that the invention will encourage bone ingrowth into a porous or biocompatiBIe implant. Thus, in an embodiment of this aspect the surgical implant and/or cement may be coated with the pro-inflammatory compound. In a further embodiment the pro-inflammatory compound may be covalently bound to the surgical implant and/or cement. This may be via direct binding of the compound of the invention to the implant surface or it may be by direct binding to a coating enveloping the implant surface, such as a biocompatiBIe polymeric material. It is envisaged that in this aspect of the invention the surgical implant may be a joint replacement, a dental implant, a plate, a screw, a pin, an intramedullary or intraosseous device or another suitaBIe implant according to the earlier aspects of the invention.

In a preferred embodiment of all aspects of the present invention, the pro-inflammatory compound is an inhibitor of indoleamine 2, 3, dioxygenase 1 (IDO). Alternatively, the pro-inflammatory compound may not be an inhibitor of IDO but it may be used in combination with an inhibitor of IDO. IDO inhibitors will be known to the person of skill in the art and any such inhibitor may be used in the present invention. Details of inhibitors of IDO may be found in Vottero et al (2006) Biotechnol. J. 1: 282-88; Yue et al (2009) J. Med. Chem. PuBIished online June 2009; Kumar et al (2008) J. Med. Chem. 51: 4968-77; Lee at al (2006) Biochemical Pharmacology 73: 1412-21; Brasianos et al, (2006) J. Am. Chem. Soc. 128: 16046-47; and Gaspari et al (2006) J. Med. Chem. 49: 684-92. Thus, the inhibitor of IDO in the present invention may be selected from, but not limited to, the group comprising 1-methyl-d-tryptophan (1-MT), 1-methyl-l-tryptophan, phenylimidazole-derivatives, hydroxyamidine chemotypes, NSC 401366 (imidodicarbonimidic diamide, N-methyl-N′-9-phenanthrenyl-, monohydrochloride) (Vottero et al. supra), 5 l (Yue et al. supra), 4-phenylimidazole (4-PI) (Kumar et al.

supra), brassinin (Gaspari et al. supra), exiguamine (Brastianos et al. supra) and rosmarinic acid (Lee et al. supra) or derivatives or analogues (synthetic or otherwise) thereof.

By “1-MT” we include the meaning that the compound comprises both the d and I enatiomers of 1-methyl tryptophan. The compound may alternatively comprise a single d or I enantiomer or a combination of both in varying degrees.

By “IDO inhibitor” we include the meaning that the compound may not only directly inhibit the activity of the IDO enzyme competitively or non-competitively, reversiBIy or non-reversiBIy, as an active site or exosite inhibitor that is effective in reducing the biological activity of IDO and related enzymes but this definition may also include any compound that inhibits the IDO enzymatic pathway, either upstream of IDO or downstream of IDO such that the effects of IDO are negated by the compound in question.

Not wishing to be bound by any theory, the inventors consider that, based on the data disclosed herein, fractures may release products such as high mobility group box 1 (HMGB1) proteins and S1001calgranulin proteins from traumatised cells. There is recent literature to suggest that in other systems, these interact with RAGE and Toll-like receptor (TLR) 2 & 4. RAGE is also an important activator of dendritic cells and it has been suggested that different ligands for RAGE have different effects.

In a possiBIe chain of events, the trauma may result in the release of HMGB1 and S100 proteins. We have demonstrated that exogenous addition of HMGB1 accelerates fracture healing (FIG. 19). Not wishing to be bound by any theory, these proteins may stimulate dendritic cells, which may then present alpha GalactoCeramide (aGalCer) with CD1d to iNKT cells, which in turn may release TNFα and other proinflammatory cytokines. This release of pro-inflammatory cytokines may act to recruit monocytes, which then release more inflammatory cytokines. Dendritic cells may also release proinflammatory cytokines.

Again, not wishing to be bound by any theory, IDO produced by the dendritic cells and iNKT cells may attenuate the response, which can be abrogated with 1-MT. IDO inhibition promotes the Th1 response, which includes interferon gamma and TNFα.

Thus, the inhibitor of IDO may be any compound that is effective in inhibiting the biological activity of enzymes that are related to IDO and/or are part of the same or complementary inflammatory pathway as IDO. The inhibitor of such related enzymes would include substances which are competitive or non-competitive, reversiBIe or non-reversiBIe, active site as well as exosite inhibitors and may be derived from natural sources or synthesised.

In an embodiment, the inhibitor of IDO is a compound that inhibits the activity of IDO by acting upstream or downstream of IDO in inflammation. By “acting upstream” of IDO we include the meaning that the inhibitor prevents the stimulation of the expression of IDO by inflammatory cells such as dendritic cells and iNKT cells. BY “acting downstream” of IDO we include the meaning that the compound acts to inhibit effectors of IDO expression, such as substrates of IDO.

It is envisaged that the inhibitor of IDO may be any analogue of tryptophan, synthetic or otherwise. Thus in one embodiment the inhibitor of IDO may be envisaged as a competitive inhibitor of the enzymatic activity of IDO protein.

Alternatively, the inhibitor of IDO may be an inhibitor of the expression of the IDO gene or of the translation of the IDO protein at any stage in its synthesis. In an embodiment, the inhibitor of IDO may be an agent that disrupts the expression of IDO. Such an agent may be selected from, but not limited to, the group comprising an RNAi or antisense molecule or a ribozyme directed to the IDO mRNA.

IDO is a tryptophan-degrading enzyme that has been identified as having potential immunoregulatory properties. A link between IDO and bone growth, formation, healing or other mechanism related to the present invention has not previously been made, or even suggested. The present inventors are the first to identify IDO as a key regulator in the process of the healing of bone fractures and this was entirely unexpected.

Historically, IDO has been recognised as a host defence mechanism of innate immune responses (Mellor (2005) Biochim Biophys Res Comm 338: 20-4). More recently, IDO expression has been identified as being essential to the maternal tolerance of their semi-allogeneic fetus. It is thought that local tryptophan depletion helps maintain pregnancies through suppression of T-cell-driven fetal rejection. Various cancer cells have been shown to express IDO and this is thought to be one possiBIe mechanism by which they evade the immune system. The cancers that have been associated with IDO expression include acute myeloid leukaemia, colorectal and endometrial cancer. Inhibitors of IDO have been considered as potential compounds for future use in cancer chemotherapy as 1-MT has been shown to have anticancer effects in mice. Vottero et al (2006) Biotechnol J. 1(3): 282-8 describes a yeast-based screen for inhibitors of IDO. Further articles cited above describe investigations into inhibitors of IDO. Such inhibitors are the focus of anti-cancer research programmes. The results described in the present Examples indicate that such compounds may also be useful for promoting bone growth, thus the present invention provides novel uses of these compounds.

The potential of IDO activation or introduction in the context of preventing organ transplant rejection and in suppressing allergies has also been postulated. Löb & Kö nigsrainer (2007) Lagenbecks Arch. Surg. 393: 995-1003 provides a review of IDO-mediated tryptophan catabolism with a particular focus on the role of IDO in cancer and transplantation immunology. Taher et al. (2008) J. Allergy Clin. Immunol. 121(4): 983-91 investigates the role of IDO tolerance induction during allergen immunotherapy.

The pro-inflammatory compound may be a pro-inflammatory cytokine or a combination of pro-inflammatory cytokines. It is intended that the pro-inflammatory cytokine may be selected from, but not limited to, the group comprising TNFα, IFNγ, IL-1β and IL-6, but the skilled person will recognise that further pro-inflammatory compounds and cytokines may be employed in the methods and uses of the present invention. For example the pro-inflammatory compound may also include long-lived derivatives of TNFα, TNFα muteins, lymphotoxin α, IFNγ, IL-1β and IL-6 (as well as other pro-inflammatory cytokines). Thus the compounds may include all molecules which can signal via TNFα receptors, including lymphotoxin α, TNFα muteins and TNFα conjugates such as those with serum albumin and IgGFc. Other proinflammatory molecules which activate intracellular signals in cells involved in the pathway for bone production would also be included. The compounds may also comprise inducers of pro-inflammatory cytokines such as Toll-like receptor ligands and ligands for the receptor for advanced glycation end products (RAGE). Examples of the latter include damage associated molecular pattern molecules (DAMPs or alarmins) such as high mobility group B1 (HMGB1) protein and S100/calgranulin family.

In addition, it is envisaged that an alternative embodiment may include the use of one or more pro-inflammatory cytokines in combination with one or more inhibitors of IDO.

The experimental data presented in the Examples would initially appear to be at odds with Hashimoto et al (1989) supra who found that TNF inhibited fracture healing. The present Examples show that TNFα promotes bone formation in a mouse model of a high-energy tibial fracture. We consider that reasons for the conflicting findings of the present study and the experiments performed in Hashimoto are the dose of TNFα administered to the animal and the site of administration. Without the benefit of the experimental data disclosed herein the skilled person would not have suspected that local administration of a physiological dose of TNFα could promote bone healing. This is a surprising finding and such experiments have not been suggested in the art previously.

In Hashimoto et al, systemic TNFα inhibited fracture healing. Also, Lacey et al (2009) suggested that proinflammatory cytokines can adversely affect bone development by mesenchymal stromal cells. Surprisingly, the findings presented herein are at odds with the findings of Hashimoto et al and Lacey et al (2009). In Hashimoto et al, the TNFα was administered intraperitoneally and it was administered at a dose of 40-400 μg per kg body weight to rats. By contrast, in the experiments disclosed herein, TNFα was administered topically at the fracture site at a dose of 50 ng per kg body weight at the time of fracture and again 24 hr later. It is not possiBIe to elucidate the amount of the large dose of TNFα that would have been present at the fracture site in Hashimoto but it may be assumed that this would have been a large amount, far in excess of the amount administered in the present studies. Thus, it is preferred that the pro-inflammatory cytokines of the present embodiment are administered at a physiological concentration locally at the site of injury and/or surgical intervention.

In a preferred embodiment, the compound of the present invention, when it is a pro-inflammatory cytokine is for administration to the patient at a dose of approximately multiples of nanograms per kg body weight. For example, for TNFα this may be up to 250 ng per kg body weight. For example, the dose may be between 1 to 400 ng/kg body weight; 2 to 300 ng/kg body weight; 5 to 300 ng/kg body weight; or 25 to 250 ng/kg body weight (or any combination of these upper and lower limits, as would be appreciated by the skilled person). Examples of possiBIe doses are 400, 300, 250, 200, 150, 100, 80, 70, 65, 60, 55, 50, 45, 40, 35, 30, 25, 20, 15, 10, 5, 2, 1 ng/kg body weight, but this may be any dose in between as would be understood by the person of skill in the art. The effective dose may be similar for other cytokines. Doses may be determined using techniques as described herein, for example in the Examples. For example, the in vitro system used in FIG. 10 may be used to determine relative potencies of different candidate compounds. This information can then be used to determine an initial dose for testing in a dose-escalation manner in appropriate patients, as well known to those skilled in the art. For example, based on the information in FIG. 10 indicating that a dose corresponding to 1 ng/kg is effective in an in vitro system with human cells, a starting dose of 50 ng/kg may be selected for use in a human patient. The skilled person will also appreciate that the effective dose may be adjusted as appropriate when pro-inflammatory cytokines are used in combination with one another. For example, when used in combination, the doses of individual cytokines may be reduced.

The actions of proinflammatory cytokines are diverse and vary according to the cellular environment that they are produced in. In the present invention, proinflammatory cytokines are aBIe to act as crucial mediators in the migration, proliferation and differentiation of pluripotential stromal cells to promote bone formation. They also act to promote bone formation by cells present locally.

In any embodiment of the present invention the patient may be selected from, but not limited to, the group comprising mammals, birds, amphibians, fish and reptiles. Exemplary mammals may be selected from, but not limited to, the group comprising humans, apes, monkeys, sheep, cattle, goats, swine, horses, dogs, cats, mice, rats, guinea pigs, hamsters, rabbits and gerbils. In a preferred embodiment of the present invention the patient is a human.

In a further aspect of the invention, the inhibitor of IDO does not necessarily have to exhibit pro-inflammatory activity. Any inhibitor of IDO activity, expression or translation may be included as a compound of this aspect of the invention regardless of any of other properties that this compound may or may not demonstrate. Embodiments discussed above in relation to aspects of the invention set out in relation to pro-inflammatory compounds are also relevant to this additional aspect of the invention, as will be apparent to those skilled in the art.

Accordingly, the present invention provides a method of promoting bone formation in a patient at a site in need thereof, the method comprising the step of administering a compound to the site, wherein the compound is an inhibitor of indoleamine 2, 3, dioxygenase 1 (IDO).

The present invention also provides for the use of a compound in the manufacture of a medicament for promoting bone formation in a patient at a site in need thereof, wherein the medicament is for administering the compound to the site, and wherein the compound is an inhibitor of indoleamine 2, 3, dioxygenase 1 (IDO).

The invention further provides an inhibitor of indoleamine 2, 3, dioxygenase 1 (IDO) for use in promoting bone formation in a patient at a site in need thereof. The inhibitor of indoleamine 2, 3, dioxygenase 1 (IDO) may be for administering at the site in the patient.

All documents referred to herein are incorporated herein, in their entirety, by reference.

The listing or discussion of a prior-puBIished document in this specification should not necessarily be taken as an acknowledgment that the document is part of the state of the art or is common general knowledge.

The invention is now described in more detail by reference to the following, non-limiting, Figures and Examples.

### FIGURES

FIG. 1: High-energy open tibial fracture showing comminution and periosteal stripping with loss of overlying soft tissue envelope.

FIG. 2: Comparing the effects of adjacent muscle and fasciocutaneous tissue on fracture healing of murine tibiae, using cortical bone content and 4-point bend testing. These data demonstrate improved healing with muscle in direct contact with the fracture compared to fasciocutanous tissue (equivalent to the closed model of skeletal injury only).

FIG. 3: Vascular density of adjacent fasciocutaneous tissue and adjacent muscle tissue over a two week period of time after fracture. These data demonstrate a higher vascular density of fasciocutaneous tissue at all time points when compared to muscle.

FIG. 4: The in-vitro pluripotential nature of cells isolated from muscle. Given the appropriate stimulus, these muscle-derived stromal cells (MDSCs) can form bone, fat or cartilage.

FIG. 5: (a) Comparison of the bone forming potential of MDSC and skin fibroBIasts cultured in osteogenic medium at 4 weeks. (b) Alkaline phosphatase (ALP) expression in MDSC (muscle-derived stromal cells), skin fibroBIasts (skin-derived cells) and pre-adipocytes (fat-derived cells) cultured in osteogenic medium, at 7 days. The results demonstrate greater osteogenic potential of MDSC compared with skin fibroBIasts or pre-adipocytes.

FIG. 6: Cell surface marker expression of MDSCs. The results demonstrate that MDSCs are mesenchymal cells, and are not haematopoietic or endothelial in origin.

FIG. 7: Investigation of the fracture-derived stimulus for osteogenic differentiation of MDSC. Supernatants derived from fractures as a result of high-energy trauma produce a significantly higher osteogenic stimulus than surgically (atraumatically) cut bone.

FIG. 8: Effects of recombinant human BMP2, BMP7 and TGF-β on alkaline phosphatase (ALP) expression in cultured MDSCs. The exogenous addition of BMP2, BMP7 (alone or in combination) and TGF-β to MDSC promotes osteogenic differentiation.

FIG. 9: Investigation of the effect of antibodies to BMPs 2 and 7, TGF-β and VEGF on the pro-osteogenic effect of fracture supernatant on MDSC. No dose-dependent inhibition of the osteogenic potential of fracture-derived supernatant was demonstrated using antibodies to BMPs 2 and 7, TGF-β and VEGF. Therefore, these growth factors are not responsiBIe for the osteogenic stimulus present in fracture-derived supernatant

FIG. 10: Effects of recombinant human IL-1β, IL-6 and TNFα on ALP expression in cultured MDSCs. The exogenous addition of pro-inflammatory cytokines to MDSC promotes osteogenic differentiation.

FIG. 11: Investigation of the effect of antibodies to IL-1β, IL-6, IL-6 soluBIe receptor, IL-10 and TNFα on the pro-osteogenic effect of fracture supernatant on MDSC. A dose-dependent inhibition of the osteogenic potential of fracture-derived supernatant was demonstrated using antibodies to IL-1β, IL-6, IL-6 soluBIe receptor and TNFα, but not IL-10, suggesting that these pro-inflammatory cytokines are responsiBIe for the osteogenic stimulus present in fracture-derived supernatant

FIG. 12: Effect of recombinant human Interferon-gamma on ALP expression in cultured MDSCs. The exogenous addition of Interferon-gamma to MDSC mildly promotes osteogenic differentiation in a dose dependent manner.

FIG. 13: The formation on bone nodules from MDSC cultured in serum supernatant with and without the addition of candidate pro-inflammatory cytokines, with osteogenic medium as a positive control. The addition of exogenous TNFα to serum supernatant promoted bone nodule formation (the final arbiter of osteogenic differentiation) in MDSC.

FIG. 14: The relative efficacy of pro-inflammatory cytokines, when added to serum supernatant, in attracting MDSC in-vitro, where (a) IL1β, (b) IL-6, and (c) TNFα. This figure demonstrates that TNFα and IL-6 act as chemo attractants for MDSC

FIG. 15: Control mice (C57BI/6) showing Faxitron radiographs and histological sections of the fracture site at time points (as specified) post injury.

FIG. 16: IL-1β receptor deficient mice showing Faxitron radiographs and histological sections of the fracture site at time points (as specified) post injury. No significant impairment of fracture healing was demonstrated compared to the controls (see FIG. 15).

FIG. 17: TNFα receptor 1 (p55) knockout (a) and receptor 2 (p75) knockout (b) mice showing Faxitron radiographs and histological sections of the fracture site at time points (as specified) post injury. The p55 receptor knockout mice show initial delay followed by accelerated fracture healing and by day 28 are equivalent to controls (see FIG. 15). The p75 receptor knockout mice show no significant impairment of fracture healing compared with controls (FIG. 15).

FIG. 18: Histological assessment of fracture healing in a C57BI/6 (wild type) mouse at day 14, with the addition of 20 μl rhTNFα in PBS at the concentrations stated (where 1 ng/ml is equivalent to 1 ng/kg). These data clearly demonstrate that the optimal dose range is narrow (around 50 ng/kg) and that at higher doses (500 ng/kg) exogenous TNFα inhibits fracture healing compared to carrier vehicle only (physiologically buffered saline—PBS).

FIG. 19: Comparison of wild type C57BI/6 mice with a group of mice where HMGB1 was administered at the fracture site immediately at the time of fracture and again 24 hr later. There was statistically significantly enhanced healing when the mice tibiae were assessed by CT scans 28 days post fracture.

FIG. 20: Indoleamine 2, 3 dioxygenase (100) knockout mice showing Faxitron radiographs and histological sections of the fracture site at time points (as specified) post injury. These sections demonstrate accelerated fracture healing at all time-points compared with the C57BI/6 control (see FIG. 15).

FIG. 21: Histological assessment of fracture healing in a C57BI/6 (wild type) mouse at day 14, with the addition of 20 μl-methyltryptophan (1-MT) in PBS at the concentrations stated (where 1 mg/ml is equivalent to 1 mg/kg). These data clearly demonstrate accelerated fracture healing following the addition of 1-MT at concentrations of 500 μg/ml and above within the range tested (0.5-5 mg/kg).

FIG. 22 Cells from muscle adjacent to fracture exhibit nodule formation A murine fracture model was performed as described. The mice were sacrificed at day 3. Radiographic images of the harvested limb were taken using a Faxitron MX-20 radiographic imager, (Faxitron LLC, Lincolnshire, Ill.) The fractured tibia is shown radiographically in A. Sham fracture is shown in B. The contralateral limb to A is also shown (C). Three fractured and 3 sham fractured mice were used. Samples of muscle and skin adjacent to the mid-point of the tibia (corresponding to the fracture site where present) were harvested. The samples were digested enzymatically and the digests plated in culture media. At 24 hours the cells were fixed using 10% formalin and stained for alkaline phosphatase using a SIGMA™ fast staining assay.

FIG. 23 Muscle-derived stromal cells exhibit tri-lineage differentiation potential Muscle derived stromal cells (MDSC) were derived from a digest of human skeletal muscle tissue (harvested with consent and ethical approval (COREC 07/Q0411/30) and processed as described in materials and methods. A: MDSC fixed with 10% formalin and stained with crystal violet, demonstrating cell morphology. B: MDSC cultured in osteogenic media for five weeks. The cells were then fixed and stained with a solution of alizarin red. C: MDSC cultured in adipogenic media for 3 weeks. The cells were then fixed and stained with oil red-O. D: MDSC pellet (containing 1×10⁵ MDSC) cultured in chondrogenic media for 3 weeks, the pellet was fixed using 10% neutral buffered formalin and paraffin embedded, then sectioned and stained with alcian BIue. The arrows demonstrate areas of cartilage formation.

FIG. 24 Comparison of the osteogenic potential of MDSC with other cell types 1×10⁴ cells of each cell type were cultured in osteogenic media for 5 weeks in triplicate. The cells were then fixed and stained as before. The images were taken at ×10 magnification and demonstrate a representative sample of a typical plate. B: 1×10⁴ cells of each cell type were cultured in osteogenic media in a 96 well plate in triplicate. At day 7 the ALP quantification assay was performed as detailed in the materials and methods section (MDSC; muscle-derived stromal cells, BMSC; bone marrow stromal cells, ADSC; adipose-derived stromal cells, SF; skin fibroBIasts). Each experiment was performed 3 times (n=3), results shown combine all experiments, ±SEM. **: P<0.01; ***: P<0.001; 1-way ANOVA with Bonferroni's multiple comparison test.

FIG. 25 The osteogenic differentiation, migration and proliferation of MDSC in response to fracture and surgically cut bone supernatants.

Fracture derived and control supernatants were produced as described in materials and methods. A: ALP expression from 1×10⁴ MDSC cultured in supernatants over 7 days, in triplicate. B: Fold-increase in migration of MDSC through an 8 μm pore membrane at 36 hours relative to serum-free media, when supernatant was placed in the lower chamber of a transwell system. C: Fold increase in cell number over 5 days, relative to starting cell number (2.5×10³ cells) when cultured in supernatant. Each figure part combines 3 experiments (n=3, each performed in triplicate), using MDSC from different donors, with the same 6 supernatants, ±SEM. *: P<0.05; **: P<0.01; ***: P<0.001; 1-way ANOVA with Bonferroni's multiple comparison test.

FIG. 26 TNF-α and IL-6 promote osteogenic differentiation of MDSC A-D: ALP quantification assays after culture of 1×10⁴ MDSC for 7 days in a 96-well plate in triplicate with: A: fracture supernatant, ±antibody neutralization of BMPs 2/4 7 and to TGF-β. The immunoglobulin isotype controls used included IgG¹ for AbBMP-2/4 and AbTGF-β, and IgG^{2B} for Ab-BMP-7. B: human serum containing culture medium with the addition of recombinant human BMPs and TGF-β. C: fracture supernatant±antibody neutralization of TNF-α, IL-6 and IL-1β. The IgG¹ immunoglobulin was used as the antibody isotype control. D: human serum-containing culture medium with the addition of recombinant human TNF-α, IL-6 and IL-1β. E: Alizarin red staining for bone nodule formation after culture of 1×10⁴ MDSC in human serum-containing media. Recombinant human cytokine at the optimal dose indicated by D, was added for the first 3 days ONLY, and the cells were subsequently cultured in media for 32 days. A representative image from a single well is shown. All experiments were performed 3 times, in triplicate (n=3) using MDSC from 3 donors. Values for A-D represent a mean from 3 experiments ±SEM, using supernatant from 3 donors in A and C. *: P<0.05; **: P<0.01; ***: P<0.001; 1-way ANOVA with Bonferroni's multiple comparison test in A and C and with Dunnett's multiple comparison test against the media only control in B and D.

FIG. 27 TNF-α and IL-6 are chemo attractants for MDSC, and promote supernatant-mediated cell migration

Migration of 1×10⁴ MDSC through an 8 μm pore transwell membrane over 36 hours, in response to fracture supernatant with the addition of AbTNF-α, AbIL-6 and AbIL-1β. The IgG¹ immunoglobulin was used as the antibody control. B: Migration in response to rhTNF-α, rhIL-6 and rhIL-1β in human serum-containing media. Results represent change in cell number, expressed as a percentage of the supernatant, or media only control. Each experiment was performed 3 times (n=3) using 3 fracture supernatants (in A). Results represent mean of 3 experiments ±SEM. *: P<0.05; **: P<0.01; ***: P<0.001; 1-way ANOVA with Bonferroni's multiple comparison test.

FIG. 28 Pro-inflammatory cytokines TNF-α, IL-6 and IL-1β do not promote MDSC proliferation

CellTiter-Glo® luminescent cell viability assay for MDSC in culture with A: TNF-α, B: IL-6 and C: IL-1β. 2.5×10³ cells were cultured in triplicate for 1, 3 and 5 days in human serum-containing media ± recombinant human cytokine at the concentration indicated. The number of viaBIe cells in culture was determined using the assay, where luminescent signal is directly proportional to the concentration of ATP from viaBIe cells. Results indicate mean ±SEM (n=3), with Dunnett's multiple comparison test against the serum-containing media control. *: P<0.05.

FIG. 29 MDSC localize to the emerging callus after injection following fracture 1×10⁵ MDSC harvested from the hind limbs of 8-week old eGFP-expressing female C57BI6 mice were injected into the pocket formed by periosteal stripping ± mid-tibial osteotomy of 10 week old female C57BI6 mice. At 7 days, the limbs were harvested, fixed in 10% formalin for 24 hours and decalcified in 10% formic acid for 7 days prior to bisection for histological preparation. One histology section is shown for each condition, and is representative of 3 limbs (n=3 for fracture and sham fracture). A: Stained with Masson's trichrome, demonstrating the histological section with the fracture site shown. B: Under fluoroscopic light, demonstrating clusters of eGFP positive cells at the endochondral margin of the forming fracture callus, and the skin wound on the fasciocutaneous surface. C: High powered image of eGFP cells within soft callus. D: and within the healing skin E: control (sham fractured) limb, demonstrating eGFP cells within the skin wound only (arrows).

FIG. 30 Data from computerised tomography scans of mouse tibial fractures 28 days post injury showing improved healing following local administration of an optimal dose of TNFα at the fracture site (50 nanograms per kg body weight) at the time of fracture and 24 hr later. IDO deficient mice (IDO^{-/-}) also show accelerated healing compared to wild type controls, as do C57BI/6 mice following administration of 1-methyl tryptophan (an IDO inhibitor) at the fracture site.

### EXAMPLE 1

### The Actions of Pro-Inflammatory Cytokines Lead to Accelerated Healing in a Mouse Model of High-Energy Fractures.

Experimental Details and Results

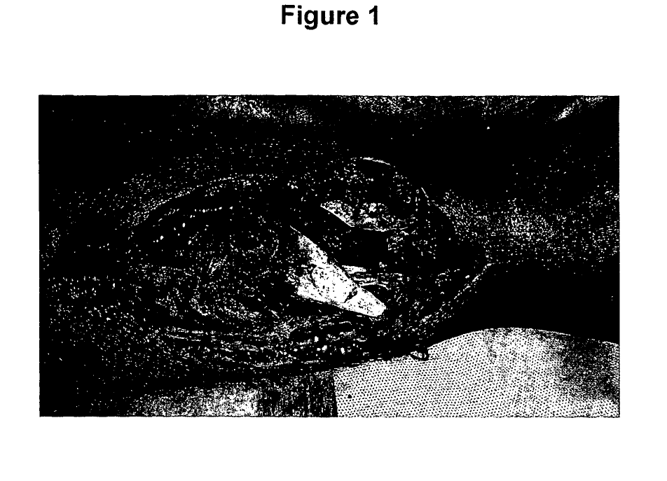

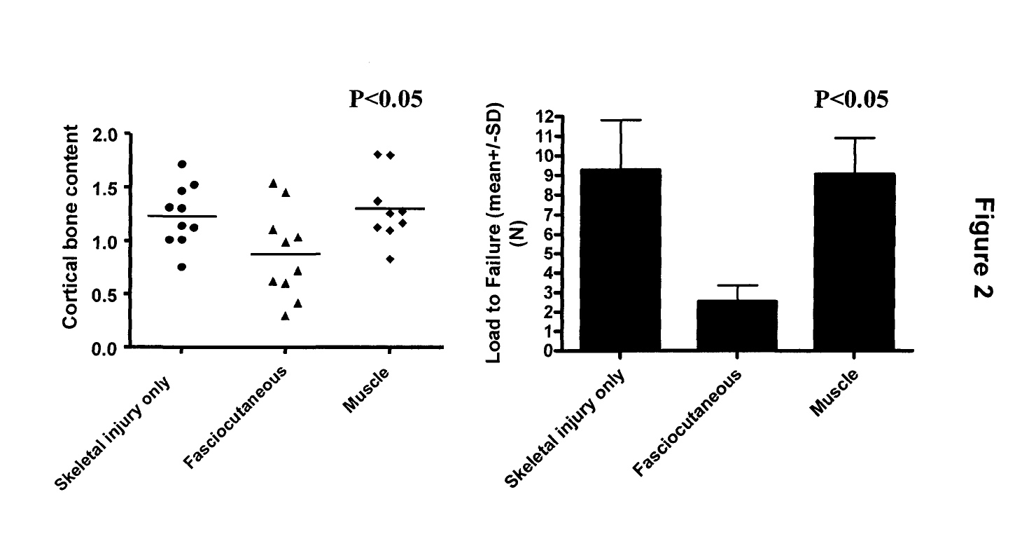

High-energy fractures are stripped of periosteum and consequently bone healing occurs by endochondral ossification. The deficient soft tissue envelope can be reconstructed using either muscle or fasciocutaneous tissue (skin with subcutaneous fat and fascia). FIG. 1 illustrates a high energy tibial fracture.

As injuries are heterogeneous and clinical variaBIes preclude adequate matched controls, we developed a novel murine open tibial fracture model to compare exclusively the effects of muscle and fasciocutaneous tissue on fracture healing in bone stripped of periosteum (Harry LE, et al. (2008) J Orthop Res. 26(9):1238-44). This is a reproduciBIe model, used in over 1500 mice by surgeons since 2002.

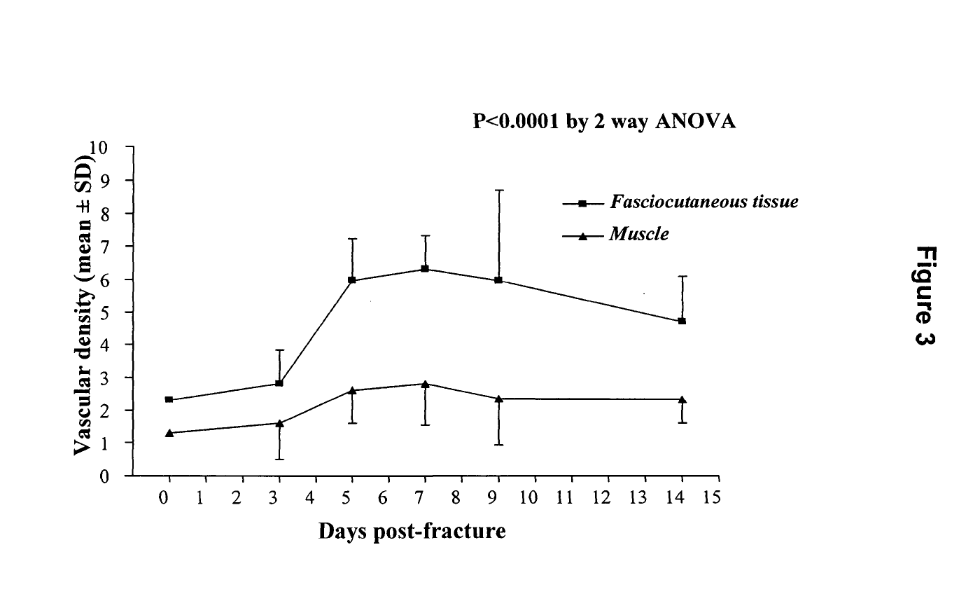

We found that direct contact of muscle with the fracture site led to accelerated fracture healing, increased new bone formation and a higher load to failure. This data is illustrated in FIG. 2. The graph on the left of FIG. 2 shows cortical bone content at 28 days. There was significantly higher cortical bone content when the fracture was covered by muscle. The graph on the right of FIG. 2 shows the results of 4 point bend testing, the final arbiter of healing strength. It demonstrates that fracture covered by muscle had a significantly greater load to failure than when covered by fasciocutaneous tissue.

These data support the finding that muscle promotes fracture healing in bone stripped of periosteum.

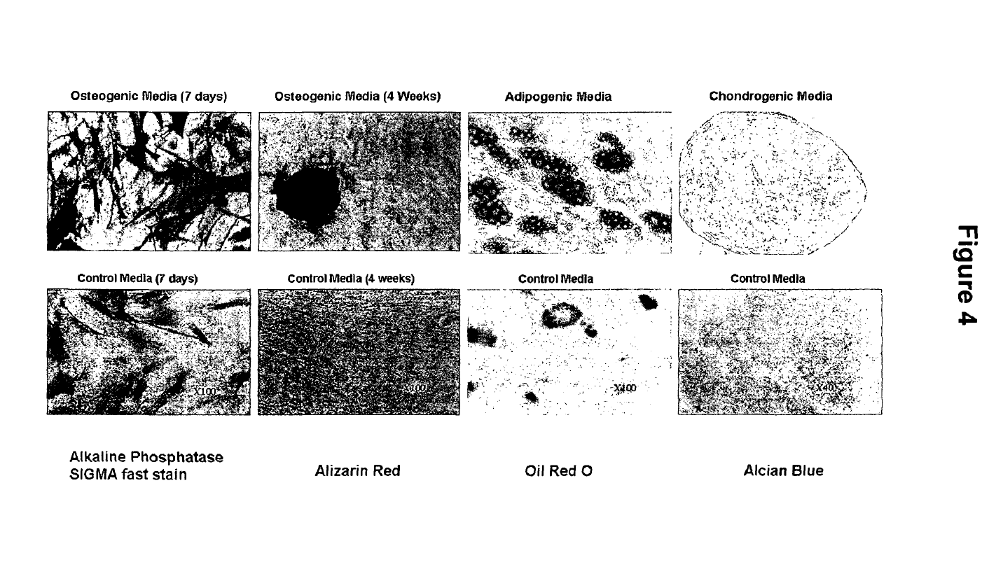

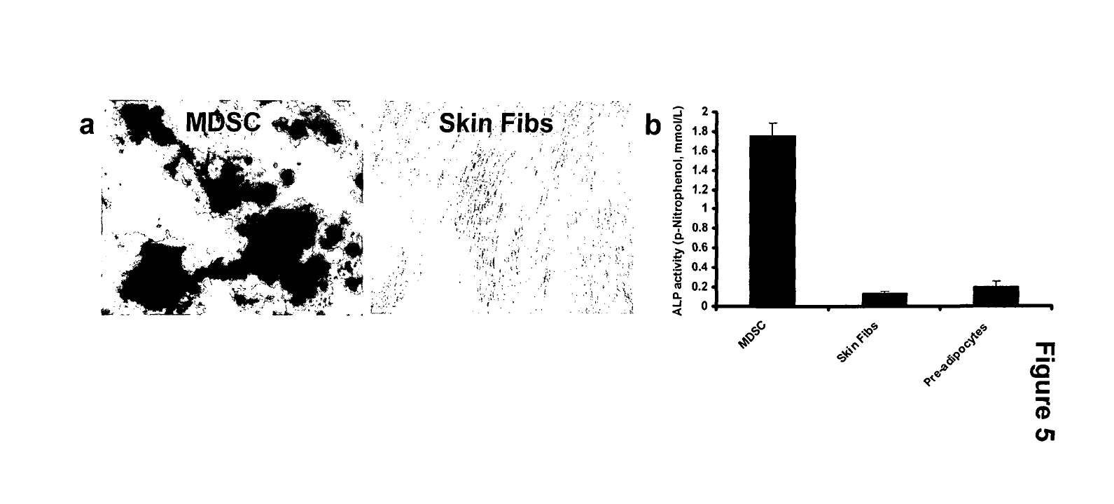

The vascular density of fasciocutaneous and muscle tissue adjacent to the fracture site was investigated and the results are displayed in FIG. 3. The vascular density of adjacent fasciocutaneous tissue was greater than adjacent muscle throughout the fracture healing period, suggesting that the mechanism by which muscle promotes fracture healing is not related to increased vascularity.

Gene expression in cells isolated from the tissue adjacent to the fracture was investigated. Cells isolated from the muscle adjacent to the fracture, but not fasciocutaneous tissue, up-regulated the osteoprogenitor genes Runx2, alkaline phosphatase (ALP) and Osterix, from as early as day 1 (data not shown). This suggests that cells resident in muscle were recruited to aid endochondral fracture healing.

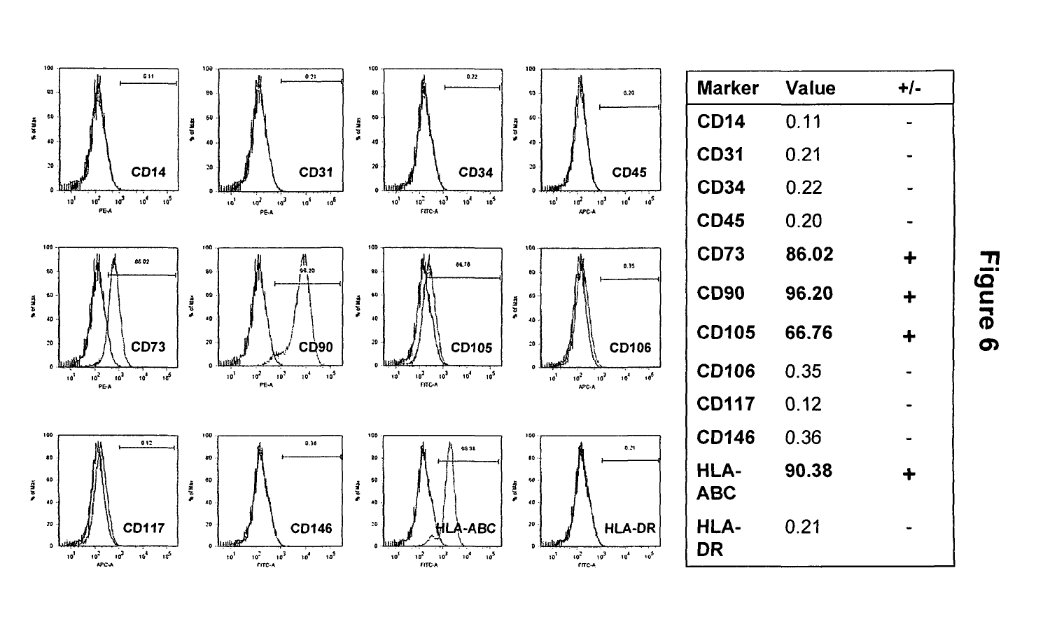

The pluripotential nature of these cells was confirmed by their ability to form bone, fat and cartilage in vitro. These results are displayed in FIG. 4.

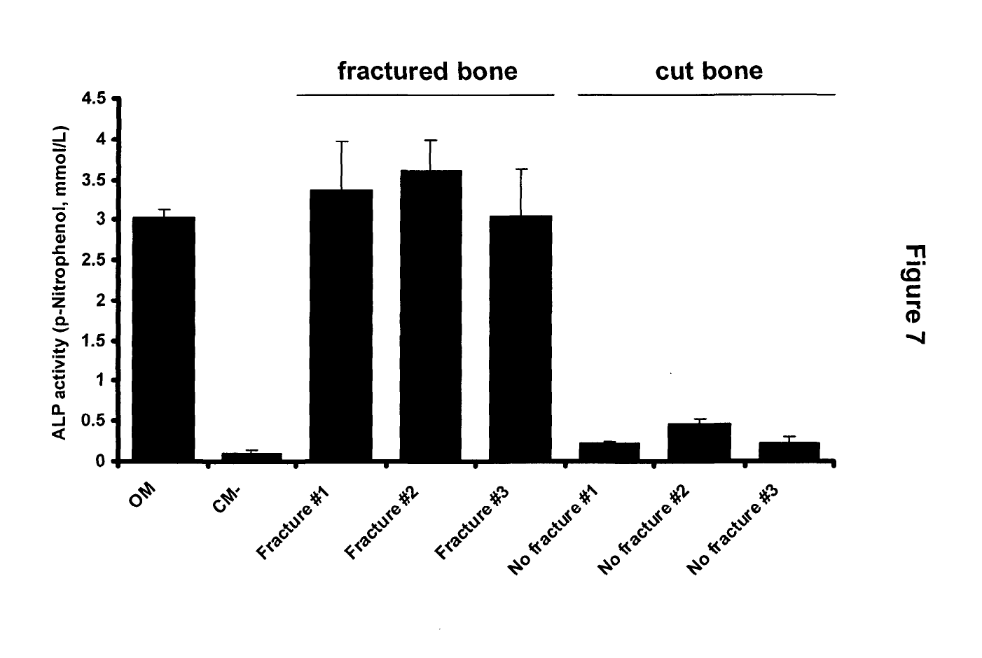

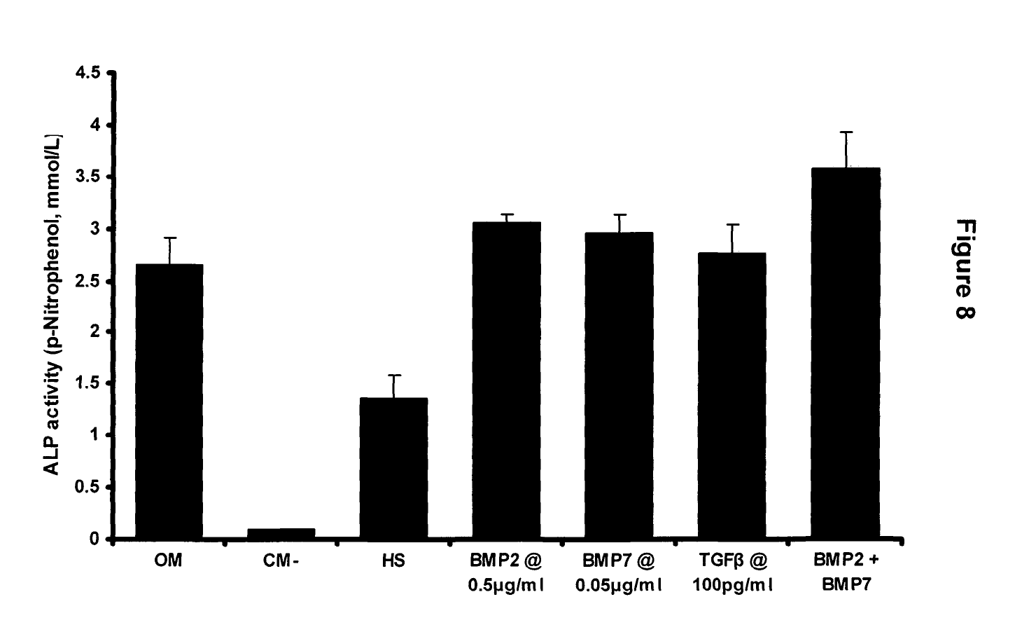

When comparing the bone forming potential of MDSC and skin fibroBIasts by culturing in osteogenic media; only MDSC formed bone nodules (FIG. 5a). Alkaline phosphatase (ALP) expression correlated with bone nodule formation (FIG. 5b). MDSC were cultured in osteogenic media for 28 days in order to attempt to culture bone nodules in vitro. Skin fibroBIasts, with simulated fasciocutaneous tissue, were used as controls.

The alizarin red stain was dissolved in acetic acid and quantified by spectrometry at 574 nm. Both osteogenic media, containing dexamethasone, ascorbic acid and beta-glycerol phosphate, and fracture supernatant, formed bone nodules and standard media did not. Interestingly, supernatants prepared using bone cut atraumatically by surgeons were inactive. However, as bone nodules take 28 days to culture, we needed an earlier quantifiaBIe marker of osteogenic differentiation and for this we used alkaline phosphatase (ALP). Here, we can see that the quantified ALP approximates that of the bone nodule quantification (FIG. 5). Alkaline phosphatase production of pre-adipocytes was also significantly lower than MDSC (FIG. 5b). Pre-adipocytes were tested as fat is known to be a rich source of pluripotential mesenchymal stromal cells.

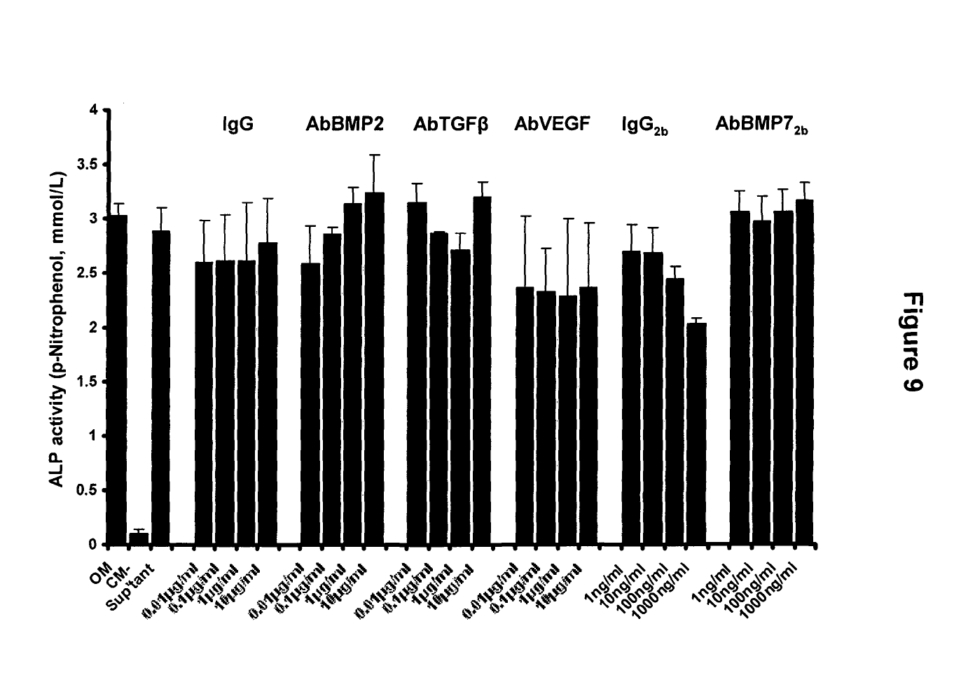

The nature of the cells isolated from muscle tissue was investigated by analysis of gene expression. Human skeletal muscle cells expressed the cell surface markers CD73, CD90, CD105 and HLA-ABC and but not CD14, CD31, CD34, CD45, CD106, CD117, CD146 and HLA-DR (data illustrated in FIG. 6). Thus, the isolated cells were mesenchymal, not haemopoetic or endothelial, in origin. Cell surface marker evaluation of the cells confirmed that they are mesenchymal stromal cells, as distinct from haematopoetic stem like cells, which are CD34 and CD117 positive.

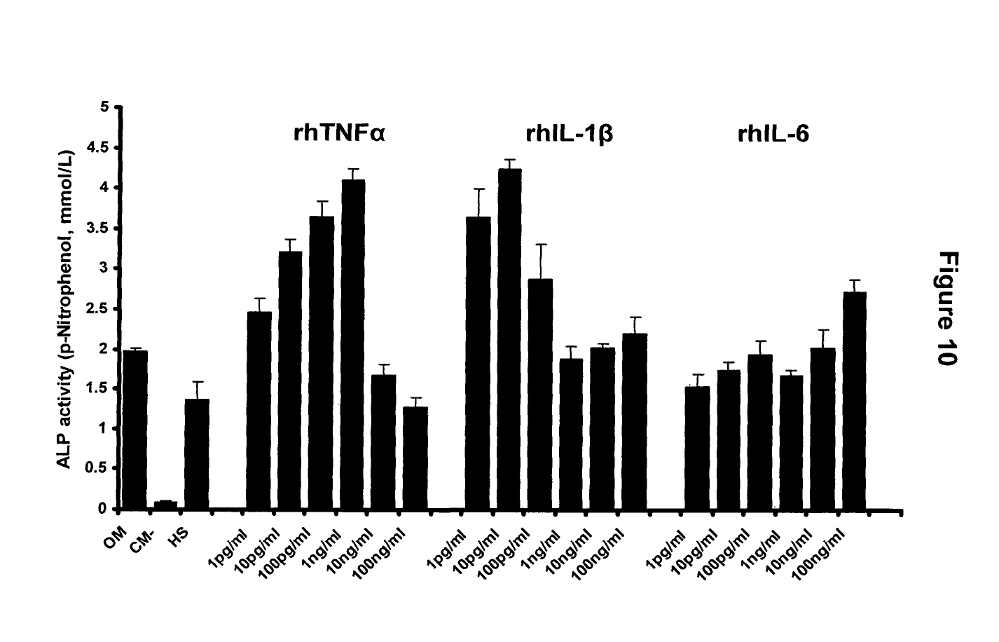

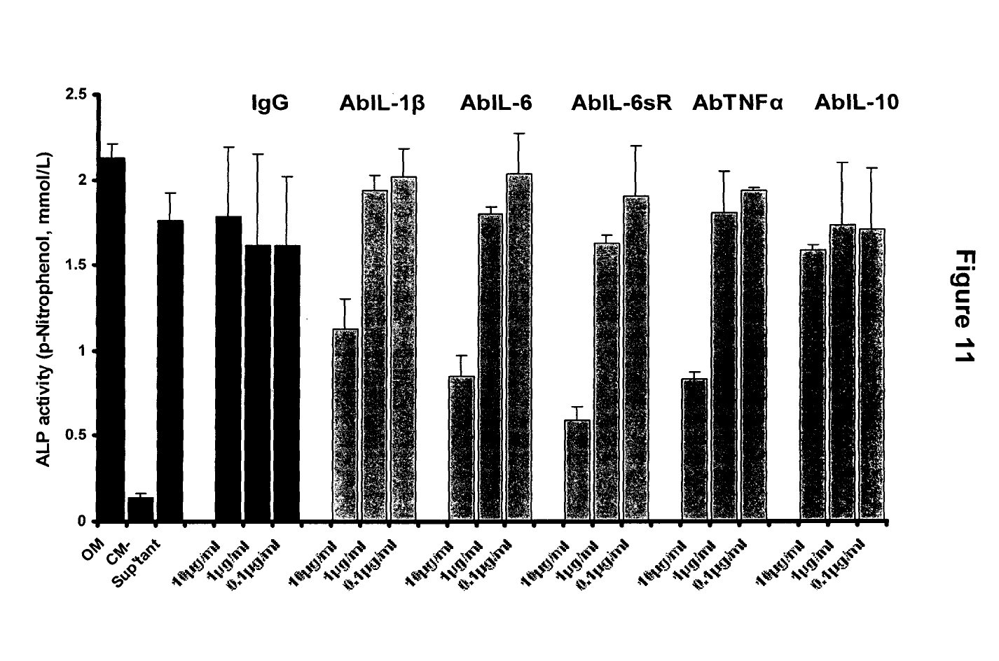

To investigate the fracture-derived stimulus for osteogenic differentiation of MDSC, fracture supernatants were prepared using human tibial fracture fragments obtained within 24 hours of injury (ethics approval number—COREC No: 07/Q0411/30). Supernatants harvested from non-fractured tibiae in patients requiring amputation as a result of severe foot trauma cut atraumatically with a fine surgical saw were used as “non-fracture controls.” Using RT-PCR and ALP quantification, we estaBIished that only fracture supernatant but not supernatants from surgically cut bone stimulated osteogenic differentiation of MDSC in vitro. The results are displayed in FIG. 7. Bone fragments were obtained from either fractured tibial fragments or non-traumatically cut bone slices. These were then incubated in serum free DMEM+Glutamax media for 24 hours. The supernatant was then filter sterilised to remove any contaminants and cellular debris. MDSC were then cultured in the presence of either control media, osteogenic media, fracture supernatant or non-fracture supernatant for 7 days before lysis and quantification of ALP levels. As shown in FIG. 7, in the presence of osteogenic media the ALP levels significantly increase above the controls. Likewise the presence of the fracture supernatant enhanced ALP levels whilst the non-fracture supernatant did not.

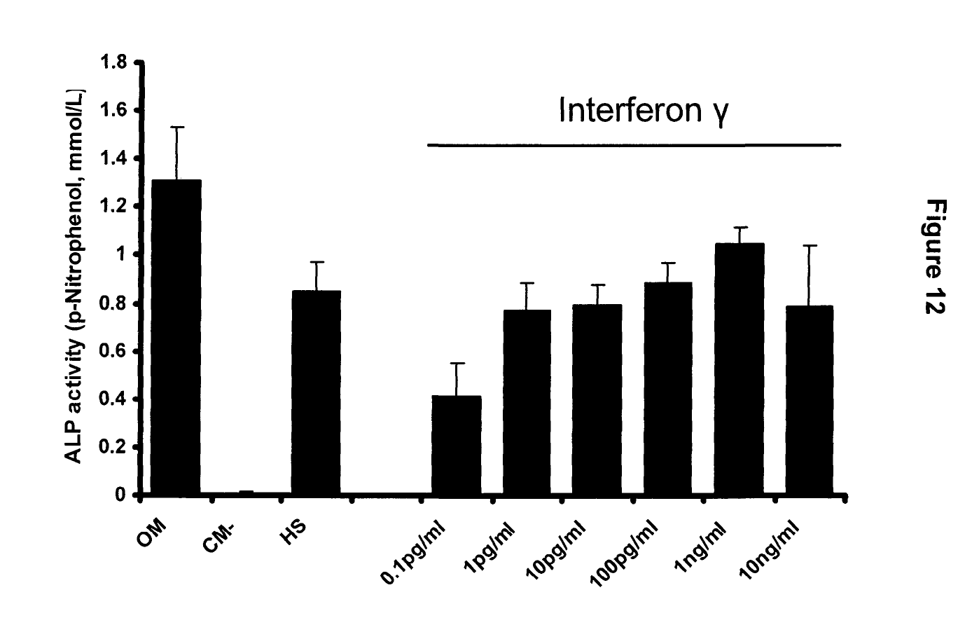

It is already well estaBIished that the bone morphogenetic proteins (BMPs) promote fracture healing and these are already used clinically. To investigate whether BMPs could promote osteogenesis by MDSC in our culture system, BMP2, 7 and TGF8 were added to MDSC. The results are shown in FIG. 8, showing that all these growth factors can promote osteogenesis by MDSC.

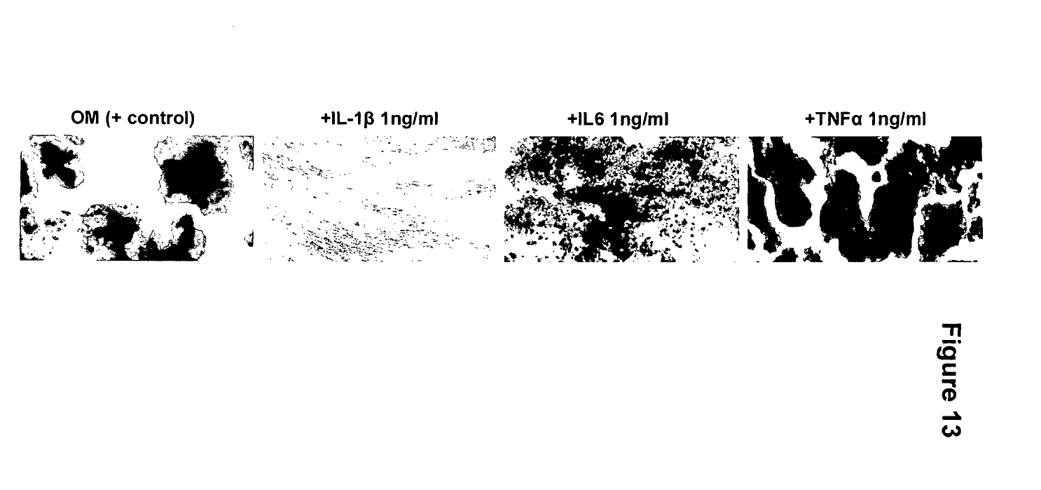

To investigate whether the pro-osteogenic effects of the fracture supernatants were due to the BMPs and VEGF, which is known to act synergistically with BMP4 (Peng H, et al. (2002) J Clin Invest. 110(6):751-9), neutralising antibodies for BMPs 2, 4, 7, TGFβ and VEGF were also added to fracture supernatant. There was no dose dependent attenuation of ALP expression (see FIG. 9).

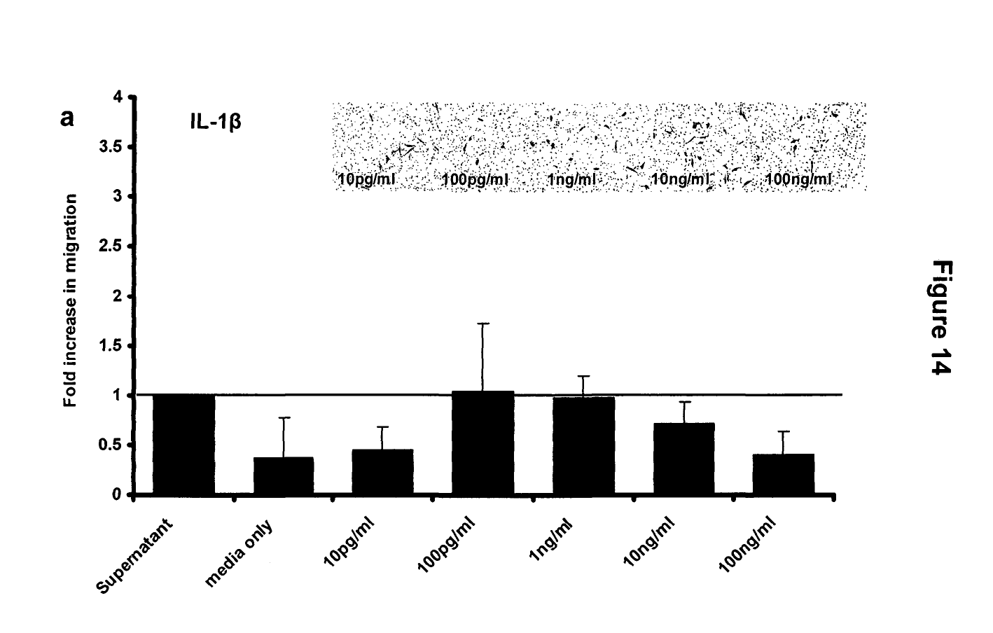

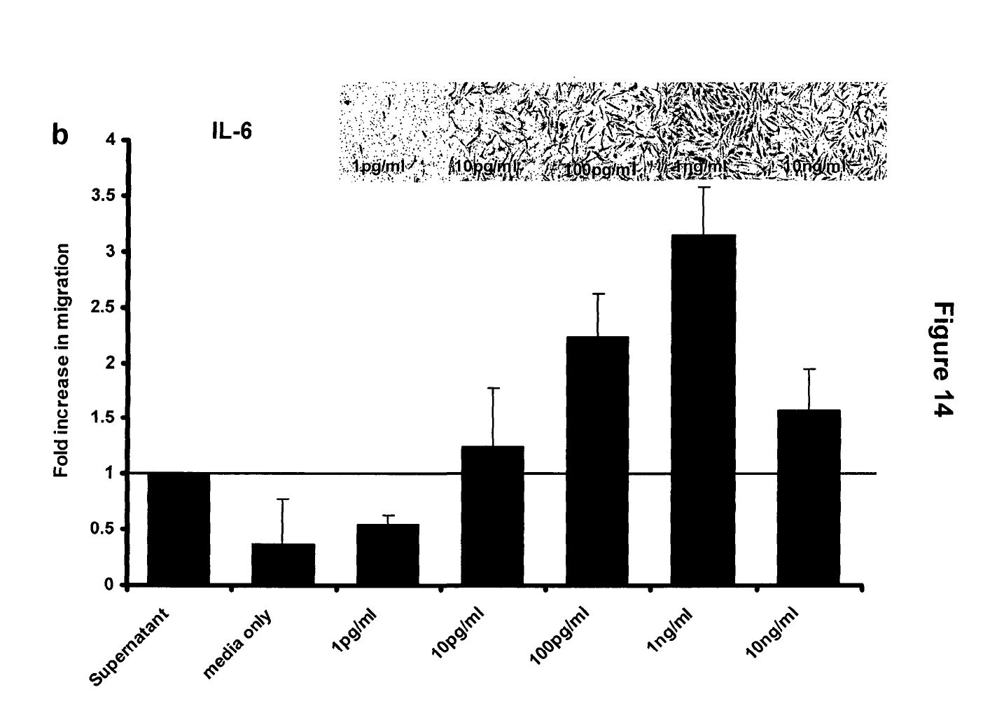

Conversely, when recombinant IL-18, IL-6 and TNFα were then added to human serum supernatant prior to culture of MDSC, there was a dose-dependent stimulation of ALP expression (see FIG. 10).

Antibodies to IL-18, IL-6 (and its soluBIe receptor) and TNFα were then added to fracture supernatant prior to culture of MDSC. Antibody to IL-10 was also used as, like IL-6, it is induced by TNFα, but is not thought to be involved in osteoprogenitor differentiation. We found a dose-dependent attenuation of ALP expression (osteogenic differentiation) using AbIL-18, AbIL-6, AbIL-6sR and AbTNFα (see FIG. 11).

These data suggested that the initial stimulus for ALP expression in MDSC by fracture supernatant was likely to be dependent on pro-inflammatory cytokine production, upstream of the BMPs.

Interferon γ (IFN-γ) is another proinflammatory cytokine that was tested in our system. There was only a modest dose-dependent stimulation of osteogenesis when added to MDSC (FIG. 12).

On adding the candidate pro-inflammatory cytokines to cultures of MDSC over a period of 5 weeks, bone nodule synthesis was promoted by TNFα and to some extent IL-6 but not by IL1β at 1 ng/ml (see FIG. 13).

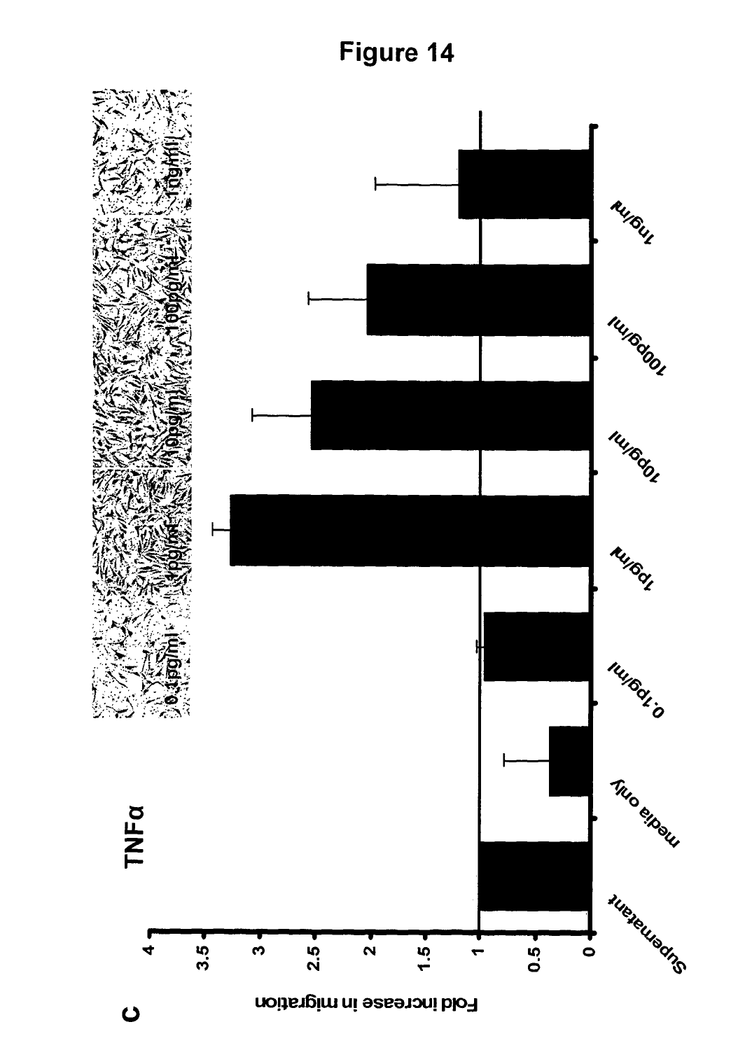

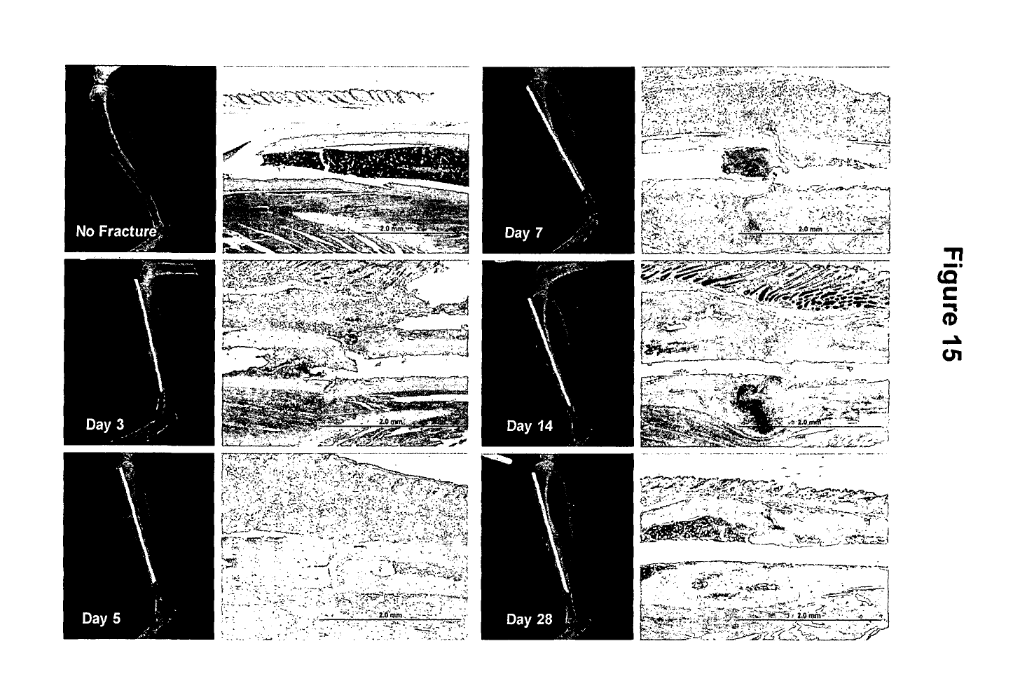

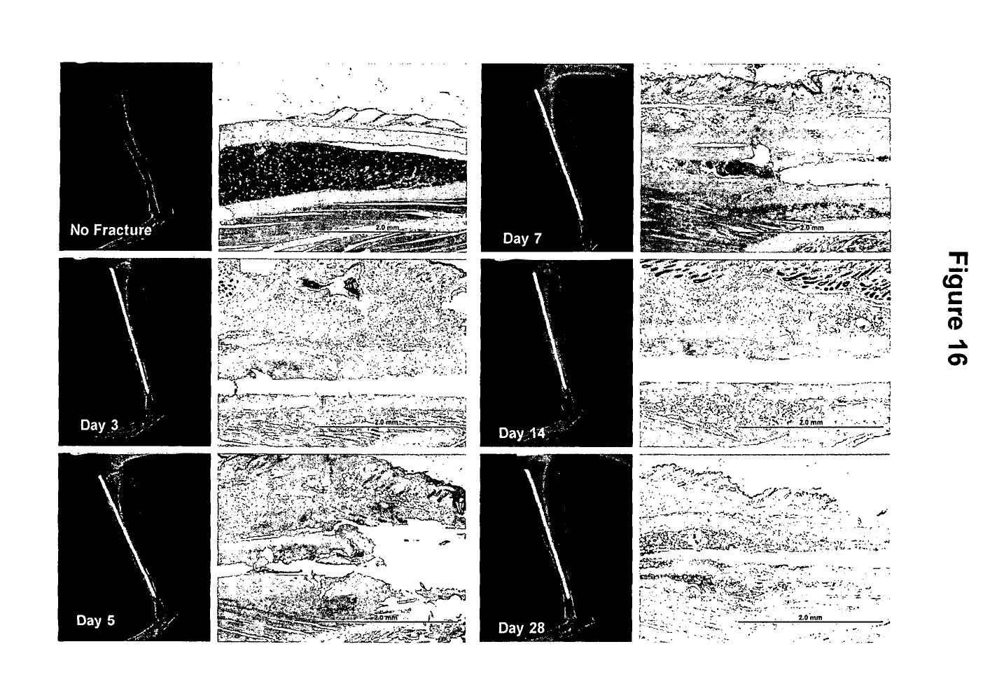

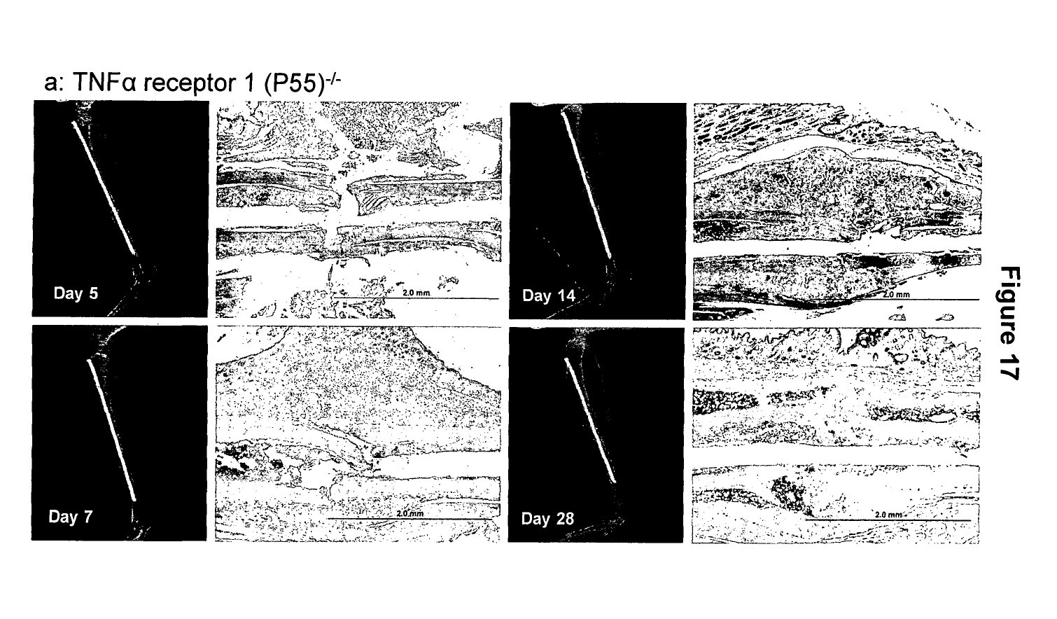

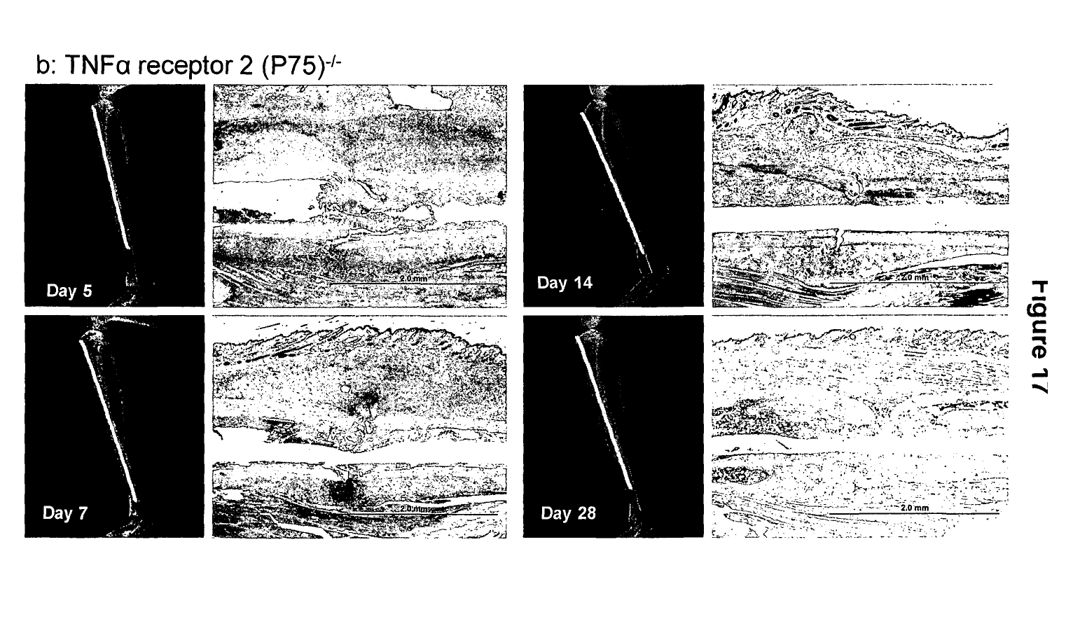

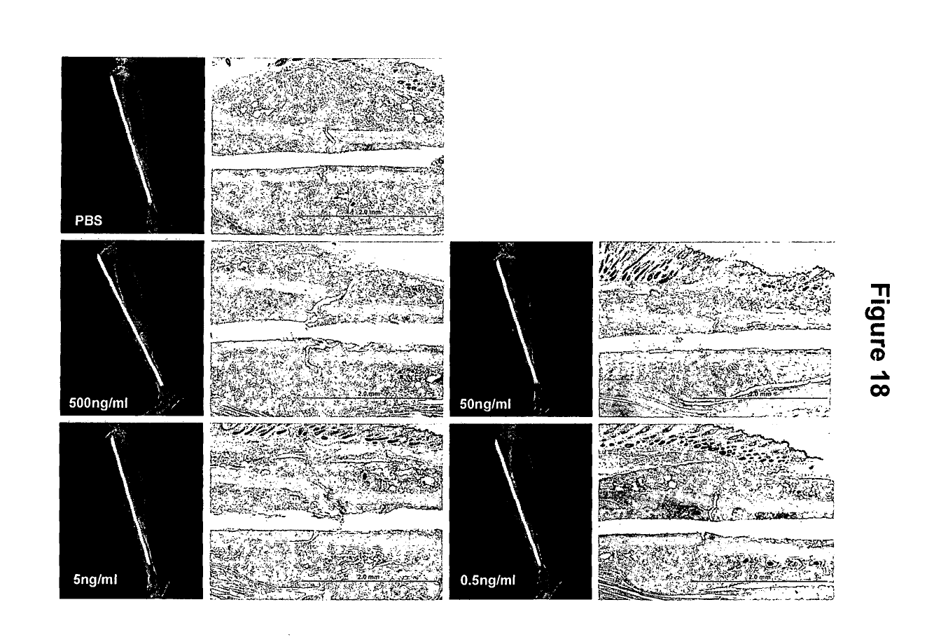

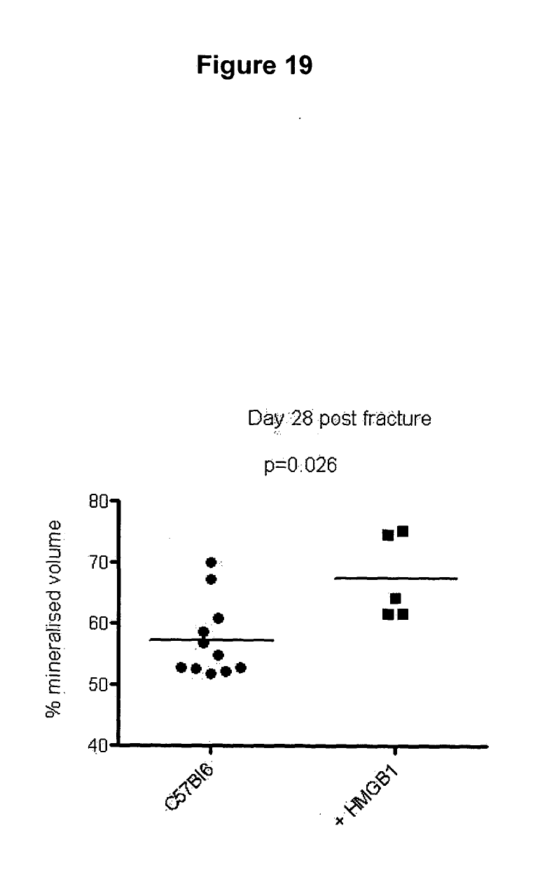

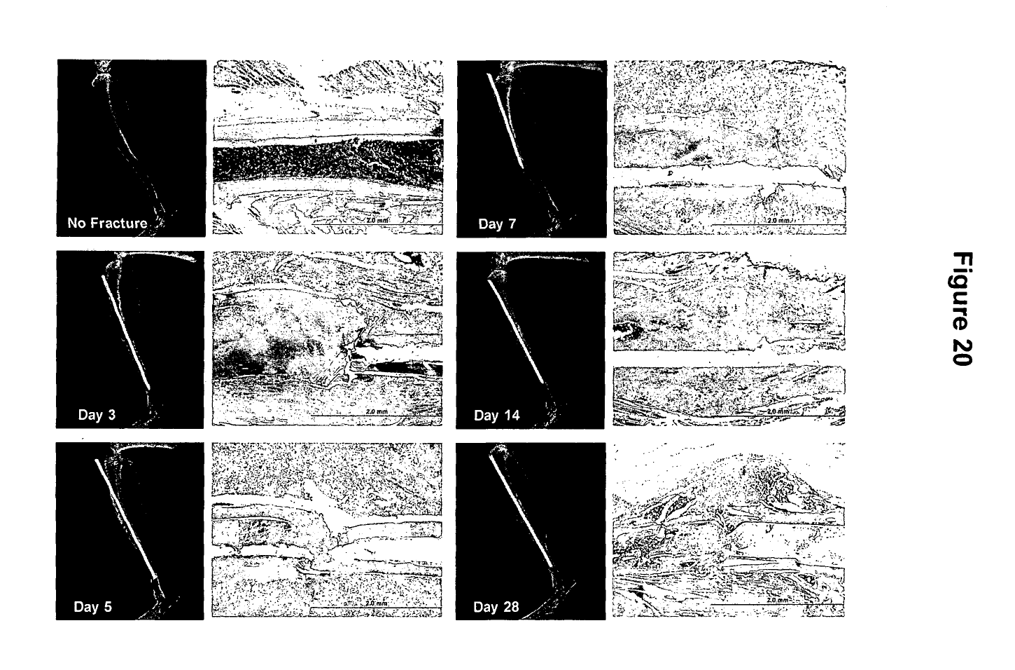

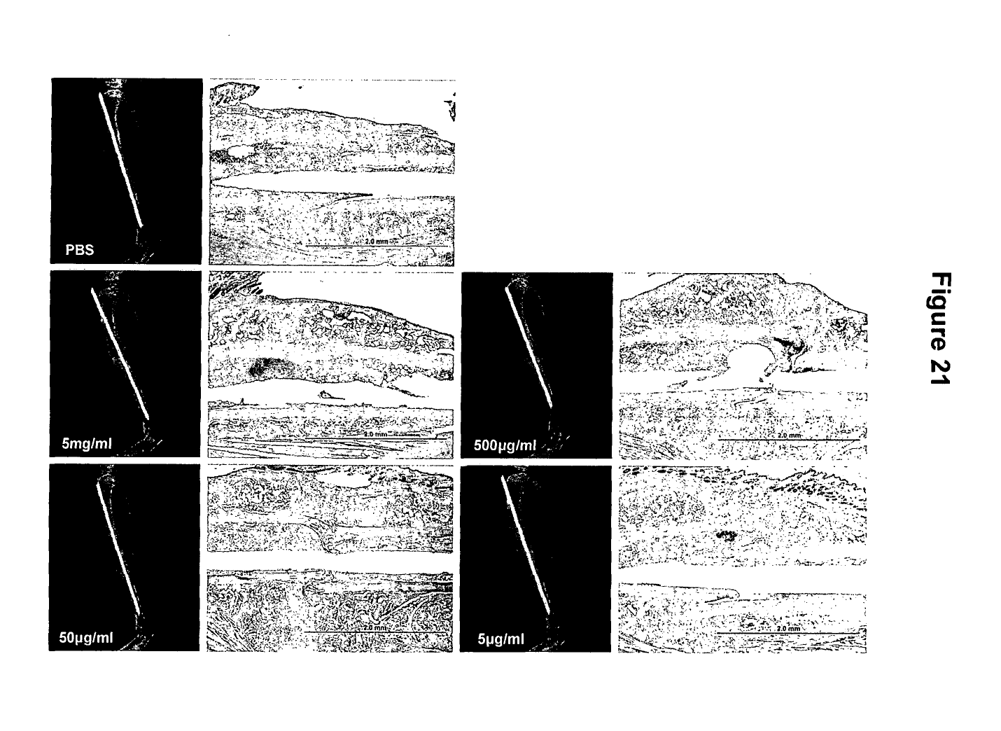

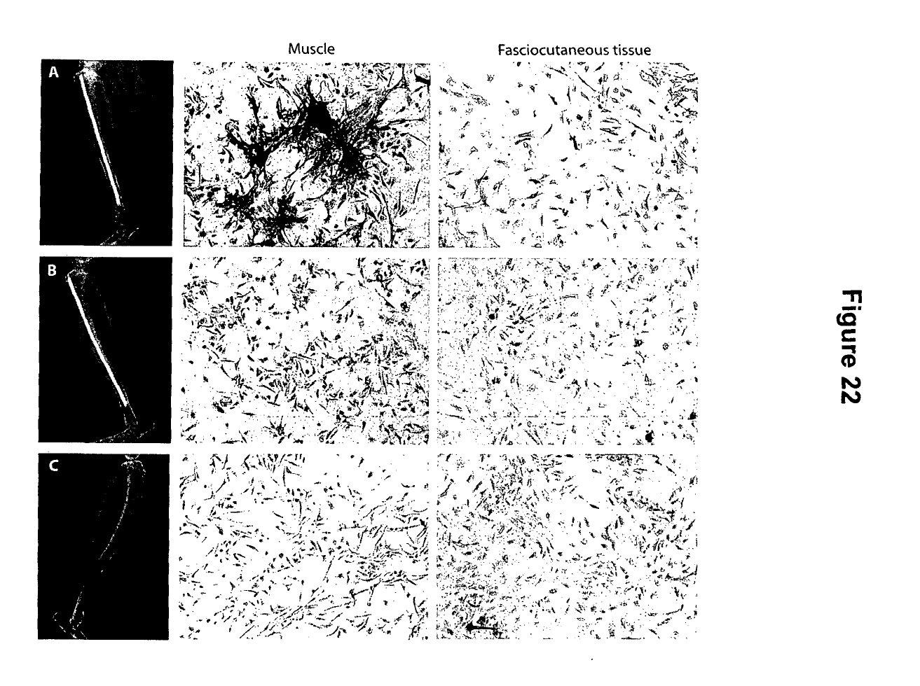

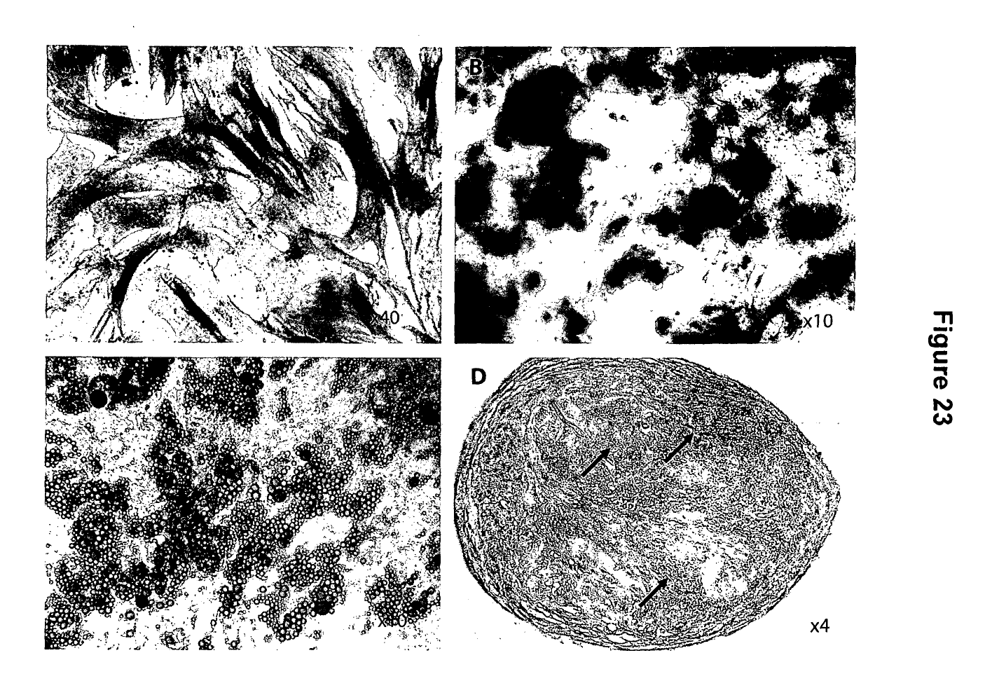

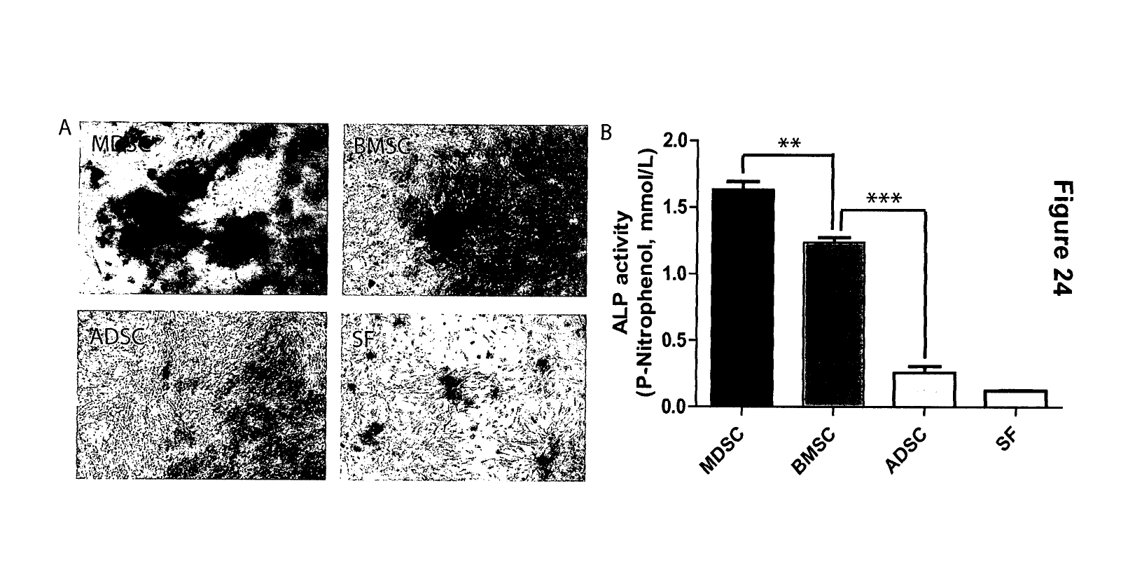

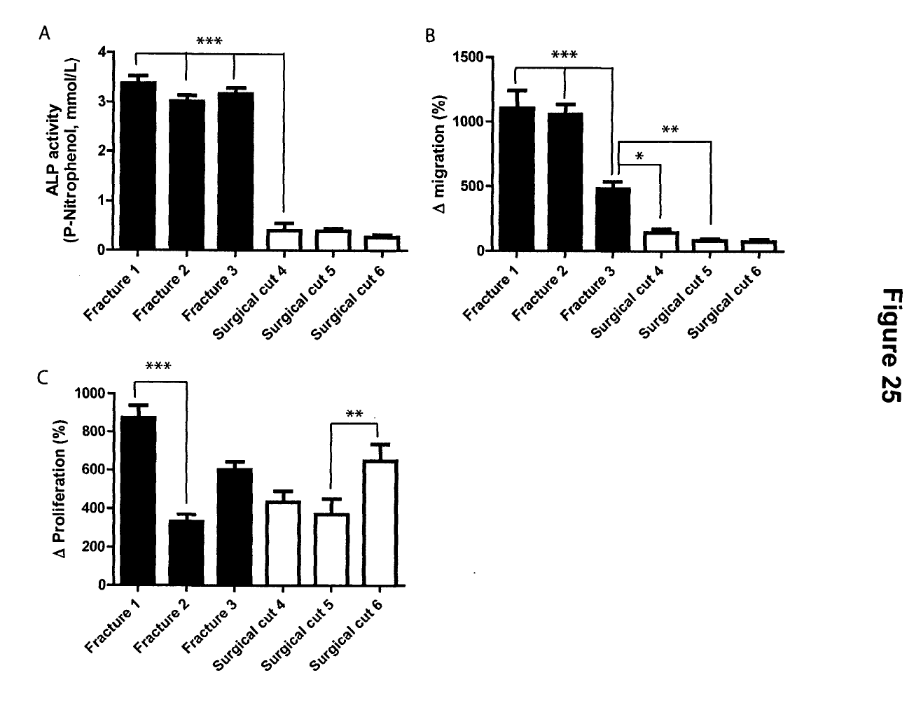

MDSC must be recruited to the fracture site from adjacent muscle. Next we assessed the efficacy of the pro-inflammatory cytokines in attracting MDSC in vitro using a transwell assay. 1×10⁴ MDSC were seeded onto a 8 μm transwell membrane in serum free media. Next day, the transwell membrane was added to a well containing 10% human serum-containing media +IL-1β, IL6 and TNFα at the end concentrations shown. At 24 hrs the membrane was cleared of any cells on the seeded side, fixed and stained. IL-1β appeared not promote MDSC chemotaxis. The results are displayed in FIG. 14 a, b and c.

These data suggest that TNFα and IL-6 but not IL-1β promote chemotaxis of MDSC. It is important to note that TNFα acts upstream of IL-6 in other systems (Charles P et al. J Immunol 163(3): 1521-8).

The effect of proinflammatory cytokines in vivo in the murine model was then tested. In the control (C57/BI) mice, the fracture healed over a period of 28 days, with faster healing seen on the side adjacent to muscle. FIG. 15 shows the histology of the fracture site over a time course of 28 days in the C57/BI control mice. At day 3 there is an inflammatory cell exudate around the fracture and by day 5 there was evidence of bone formation under the stripped periosteum at a distance from the fracture site. By day 7 there was soft callus formation around the fracture site and by day 9 there was bridging cartilage on the muscle side. There was evidence of endosteal bone formation within the medullary canal. By day 14 the was still not complete bridging by hard callus, even on the muscle side. By day 21, bone had bridged the fracture gap on the muscle side and this had occurred on the fasciocutaneous side by day 28.

### Results and Discussion

In high energy open tibial fractures stripped of periosteum, the bone heals by endochondral ossification and the soft tissue envelope can be reconstructed using either muscle or fasciocutaneous tissue. We developed a murine model to emulate these injuries and found that muscle in direct contact with the fracture led to accelerated fracture healing and 3 fold stronger union compared to fasciocutaneous tissue, (Harry L E, et al. (2008) J Orthop Res. 26(9):1238-44) despite the fact that the vascularity of the latter was greater at all time points (Harry L E, et al. Soft tissue reconstruction of open tibial fractures: an in vivo study of the effect of vascularity on fracture healing. Plast Recon Surg: In Press).

It is estaBIished that normal skeletal muscle contains pluripotential cells (muscle derived mesenchymal stromal cells—MDSC), which are capaBIe of forming bone. Our experimental findings would suggest that these are the major contributor to endochondral fracture healing as the marrow derived MSC were present equally in the muscle and fasciocutaneous groups in our murine model. Furthermore, MDSC exhibited 16-fold greater bone nodule formation in vitro than skin fibroBIasts when exposed to osteogenic medium. FACS analyses of cell surface markers showed that the MDSC were mesenchymal and not haemopoietic in origin.

To investigate the fracture-derived stimulus for osteogenic differentiation of MDSC, fracture supernatants were prepared using human tibial fracture fragments obtained within 24 hours of injury. Supernatants harvested from non-fractured tibiae in patients requiring amputation as a result of severe foot trauma cut atraumatically with a fine surgical saw were used as “non-fracture controls.”

We found that only fracture supernatant stimulated osteogenic differentiation of MDSC in vitro. Neutralising antibodies for BMPs 2, 4, 7, TGF and VEGF added to fracture supernatant had no dose dependent attenuation of MDSC osteogenesis. Conversely, there was a dose dependent inhibition of osteogenesis with neutralising antibodies to the proinflammatory cytokines, especially TNFα. Furthermore, addition of TNFα at physiological concentrations (1 ng/ml) (but not IL-1 or IL-6) led to pronounced bone nodule formation by MDSC in vitro. TNFα at this dose also promoted migration of MDSC across a Transwell membrane and led to proliferation of MDSC.

We examined the healing of fractures over a 28 day time course in IL-1β receptor knockout (FIG. 16), p55 TNFα (FIGS. 17a) and p75 TNFα (FIG. 17b) receptor knockout mice. There was no impairment of fracture healing in the IL-16 receptor knockout, and the p75 TNFα receptor knockout mice. In the p55 TNFα receptor knockout mice, there was an initial delay followed by accelerated fracture healing so that by day 28 there was no significant difference when compared to control mice. These findings would be consistent with the previously puBIished data on p55^{-/-}p75^{-/-} animals (Gerstenfeld L C, et al. (2003) J Bone Mineral Res. 18(9):1584-92).

Conversely, as can be seen in FIG. 18, exogenous addition of the short-lived cytokine TNFα at the fracture site led to accelerated fracture healing in C57BI/6 mice in a dose—dependent manner compared to control C57BI/6 mice. Over a log dose range, we found that the optimal dose was 50 ng/ml, with 5 ng/ml having a less pronounced effect whilst 500 ng/ml was inhibitory. Therefore, TNFα accelerates fracture healing at a relatively low dose, which has not been previously tested, and within a relatively narrow dose range. We are currently examining half log dose range to better define the optimal concentration.

### EXAMPLE 2

### The Absence of Idoleamine 2, 3 Dioxygenase 1 (Ido1), or Reducing its Activity, Leads to Accelerated Fracture Healing

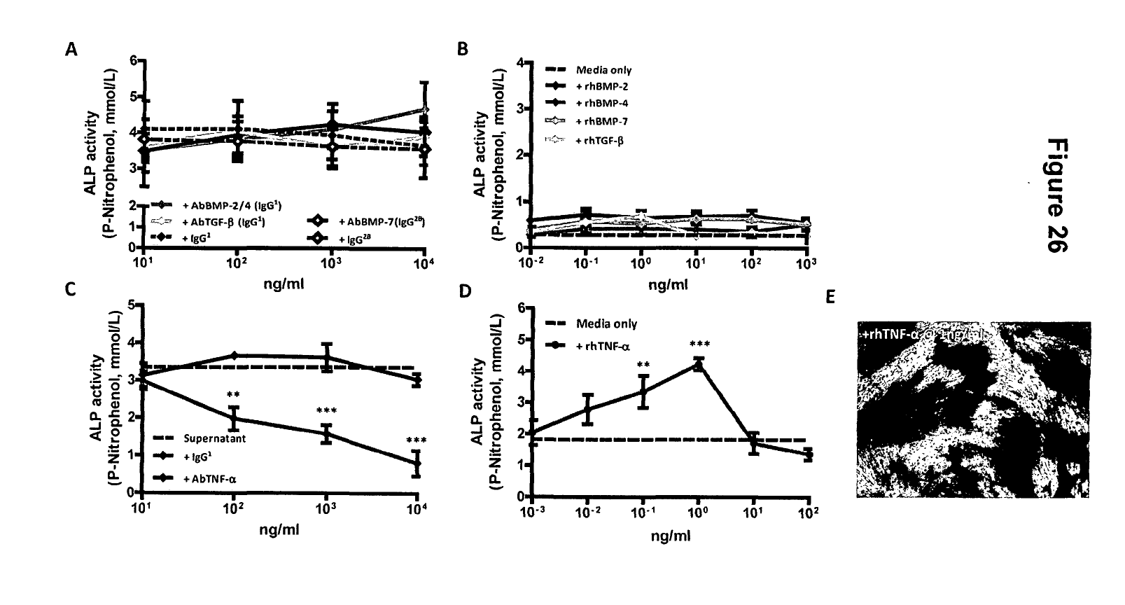

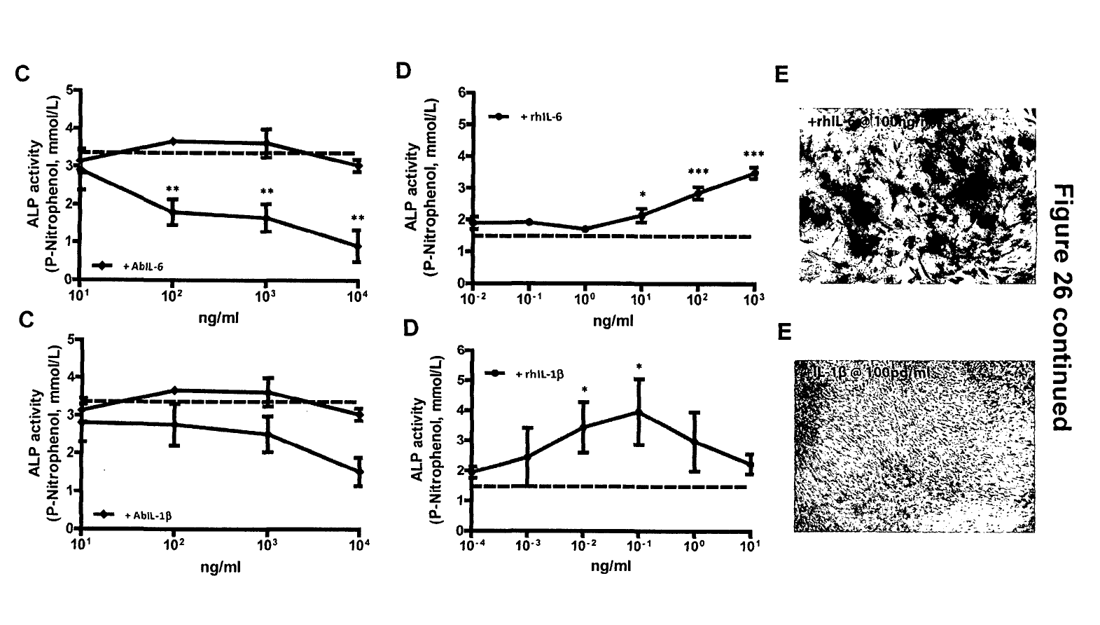

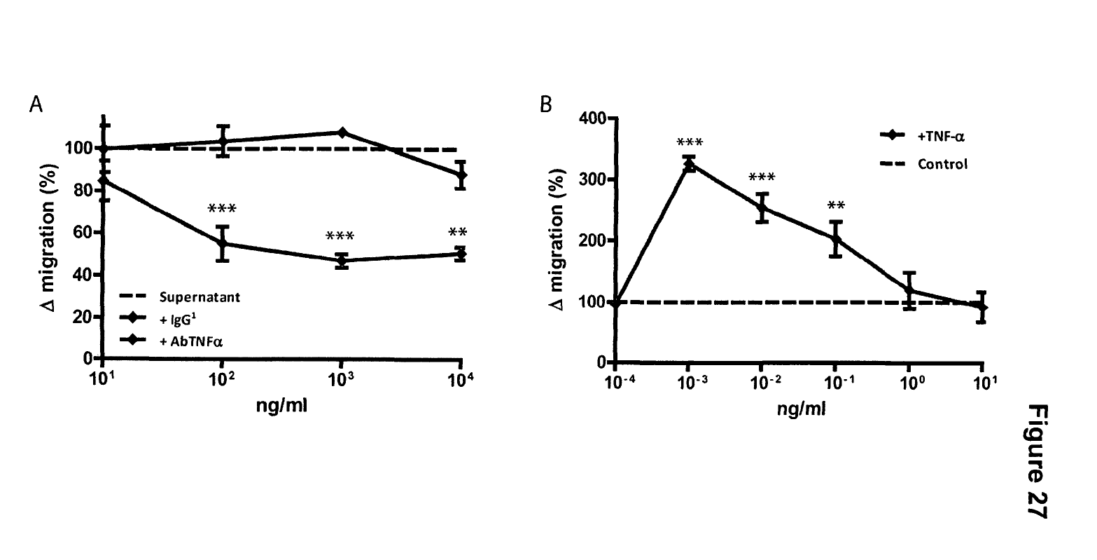

Using idoleamine 2, 3 dioxygenase 1 (IDO1) deficient mice to study the effect of an exaggerated inflammatory response, we found accelerated fracture repair compared to the C57/BI control mice (see FIGS. 20 and 21).

FIG. 20 shows that there was an intense inflammatory cell infiltrate at day 3 and at day 5, there was soft callus bridging the fracture site on the muscle side, with some ossification. By day 7 there was a large volume of soft callus on the fasciocutaneous side and almost complete bone bridging on the muscle side. At day 14, there was complete bone bridging on the muscle side and almost complete on the fasciocutaneous side. Radiologically, the fracture had remodelled at this time point.

We then tested the effect of an IDO inhibitor (1 methyl tryptophan) on fracture healing in mice. FIG. 21 shows a dose dependent acceleration of fracture healing by the addition of 1-methyl tryptophan, with the greatest response seen at the maximal dose tested (500 μg/ml)). 1-MT is poorly soluBIe in aqueous solutions and we will explore further ways of delivering higher concentrations as well as other more potent IDO inhibitors.

FIG. 30 shows data from computerised tomography scans of mouse tibial fractures 28 days post injury showing improved healing following local administration of an optimal dose of TNFα at the fracture site (50 nanograms per kg body weight) at the time of fracture and 24 hr later. The IDO knockout mice (IDO −/−) heal significantly better than the controls. When we added an exogenous IDO inhibitor (1 methyl tryptophan—1MT) we found a trend to improved healing but did not reach statistical significance. As mentioned above, 1MT has poor aqueous solubility. The use of a more soluBIe inhibitor of IDO, such as those detailed in the above description, may address the proBIems of the low solubility of 1-MT.

### EXAMPLE 3

### Tnfα Promotes the Recruitment and Differentiation of Muscle-Derived Stromal Cells for Fracture Healing

Here, using our murine model and an ex vivo model based on human tissue, we have investigated muscle derived stromal cells as a source of osteoprogenitors for bone formation and have sought evidence for their recruitment and differentiation following fracture in vivo. In particular, we investigated the influence of the early fracture environment, the pro-inflammatory cytokines and the BMPs, on these cells. Our data show that muscle-derived stromal cells readily form bone in vitro when stimulated by supernatant derived from fractured, but not surgically cut bone. The stimulus was inhibited by antibodies to TNF-α and IL-6, RhTNF-α and rhIL-6 promoted de novo bone formation and cell migration. The chemo attractant effect of supernatant muscle-derived cells was also attributed to TNF-α, which exerted the more potent effects on both differentiation and migration. Moreover, the fracture environment was shown to promote the migration of MDSC in vivo.

Results

A Population of Osteoprogenitor Cells can be Isolated from Muscle Adjacent to Fracture

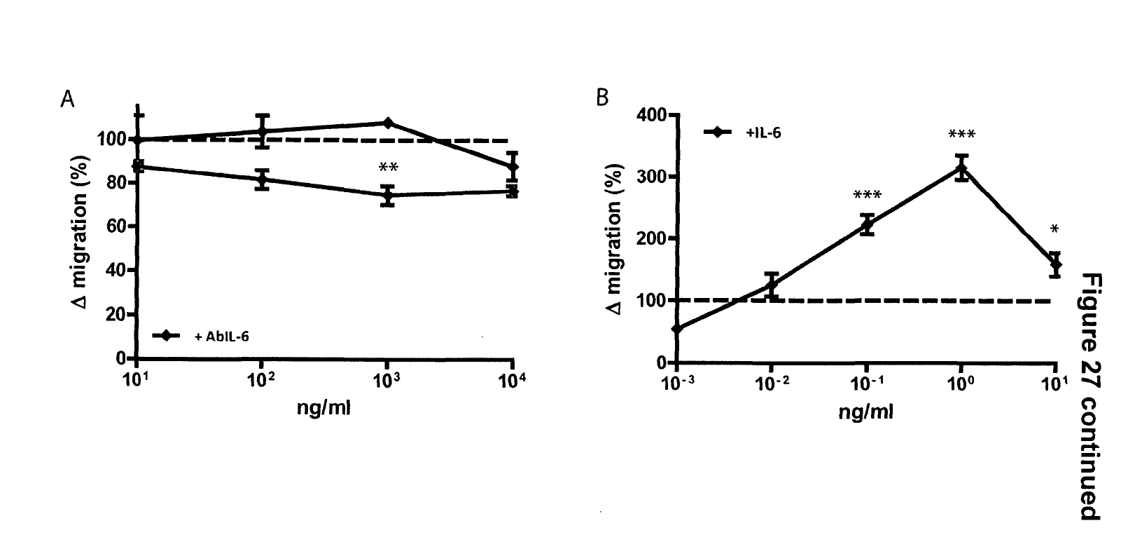

The influence of fractured and un-fractured bone on adjacent muscle was investigated using our mouse model of a periosteally-stripped (high-energy) open tibial fracture (Harry et al '08). Mice underwent right tibial cannulation and intramedullary pin fixation, mid-diaphyseal osteotomy, periosteal stripping and were sacrificed at day 3 following the surgical fracture. Cells were isolated from the muscle adjacent to the tibia posteriorly and from the skin adjacent to tibia anteriorly. The contra-lateral un-fractured limb was used to obtain control tissues. The tissue samples were digested enzymatically and the isolated cells were fixed and stained for alkaline phosphatase (ALP) expression, a surrogate marker of early osteogenic differentiation. Microscopy revealed that cells isolated from muscle adjacent to fracture strongly expressed ALP, but not cells isolated from the ipsilateral fasciocutaneous tissue or from the muscle of the contralateral leg. Furthermore, ALP expressing cells were not seen in sham fracture controls, which were subject to periosteal stripping and soft tissue dissection but no tibial osteotomy. This suggested that the fracture, as opposed to the soft tissue dissection, led either to the osteogenic differentiation of cells residing locally within skeletal muscle or promoted the systemic recruitment of osteoprogenitor cells (FIG. 22).

Human Skeletal Muscle Contains a Stromal Cell Population with Tri-Lineage Stem Cell Characteristics

While our murine model provided a valuaBIe in vivo system, results from rodent models do not always readily translate to clinical practice. Therefore, subsequent in-vitro studies were performed using human tissue. Samples of healthy skeletal muscle obtained from sites remote to the limb fracture, and hence not pre-conditioned by the fracture environment, were obtained from off-cuts of reconstructive free muscle flaps. Muscle-derived stromal cells (MDSC) were isolated following enzymatic digestion of the tissue as detailed in materials and methods. The MDSC were characterized using flow cytometry and shown to express CD73, CD90, CD105 and HLA-ABC but not CD14, CD31, CD34, CD45, CD106, CD117, CD146 and HLA-DR, thus confirming that they were of the mesenchymal, and not haematopoietic or endothelial lineage (TaBIe 1). This profile of cell surface markers is observed in numerous stromal populations, whether they possess stem-like properties or not, and is in agreement with our earlier work (Jones et al., 2007).

TABLE 1
Phenotypic analysis of the MDSC cell population

| Cell surface marker | % positive cells | ± SEM |
|---|---|---|
| MHC 1 | 90.53 | 5.02 |
| CD14 | 0.14 | 0.14 |
| CD31 | 0.59 | 0.54 |
| CD34 | 0.46 | 0.62 |
| CD45 | 0.83 | 0.81 |
| CD73 | 85.59 | 3.14 |
| CD90 | 95.65 | 3.03 |
| CD105 | 64.88 | — |
| CD106 | 0.22 | 0.09 |
| CD117 | 0.39 | 0.47 |
| CD146 | 0.71 | 0.52 |

TaBIe 1 MDSC were assessed for their surface expression of various phenotypic markers. 1×10⁵ cells per tube were stained with PE-conjugated anti human CD14, CD31, CD73 and CD90; FITC-conjugated anti human MHC I, CD34, CD105 and CD146; APC-conjugated anti human CD45, CD106 and CD117. Values represent percentage of cells positive for the surface expression of each marker ±SEM gated against the appropriate isotype control, with a background of typically less than 1% (n=4, including 2 from 1 donor with 12 cell passages between samples)

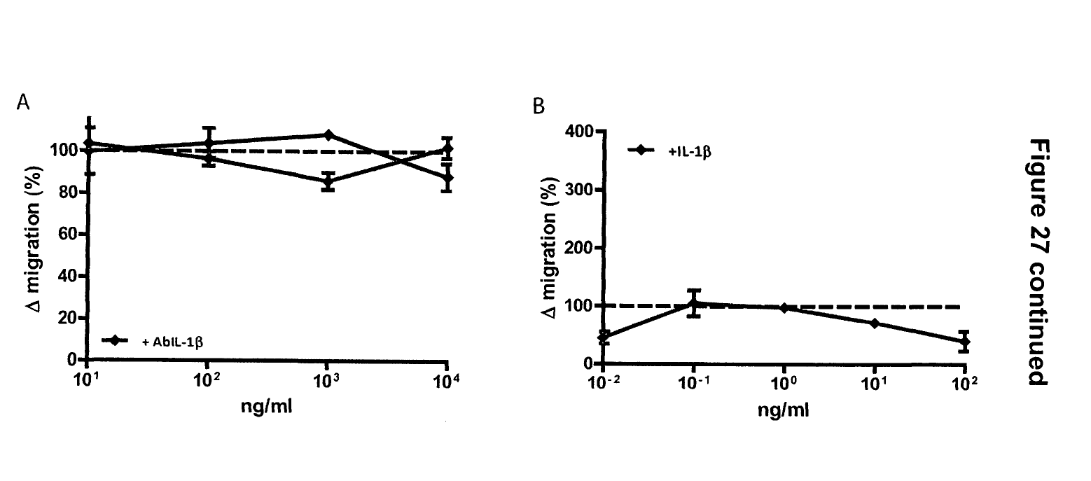

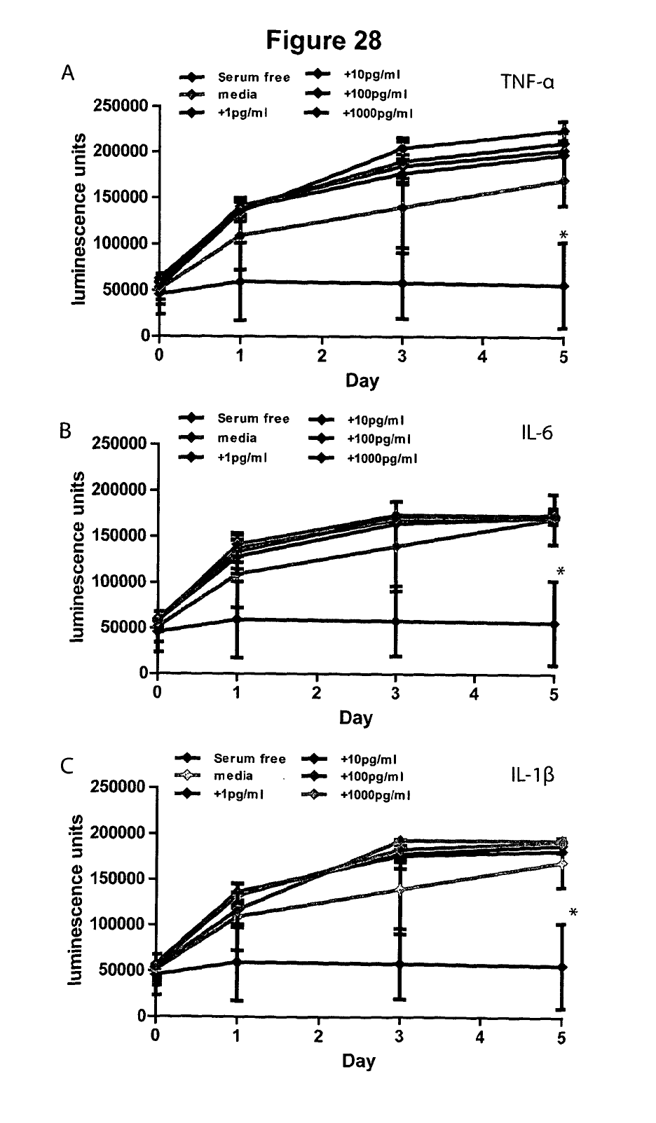

The stem-like potential of this MDSC population was investigated by assessing their ability to differentiate in vitro. MDSC were cultured in osteogenic differentiation media, adipogenic differentiation media (induction and maintenance) or chondrogenic differentiation media. The morphology of the MDSC is demonstrated in FIG. 23A. These cells readily formed bone nodules in osteogenic media, as demonstrated by alizarin red staining (FIG. 23B), fat droplets, as demonstrated by oil red-O staining (FIG. 23C) and cartilage, demonstrated by alcian BIue staining of a paraffin-embedded cell pellet (FIG. 23D).

The bone Forming Potential of Muscle Derived Stromal Cells is ComparaBIe to that of Marrow Derived Stromal Cells in Vitro

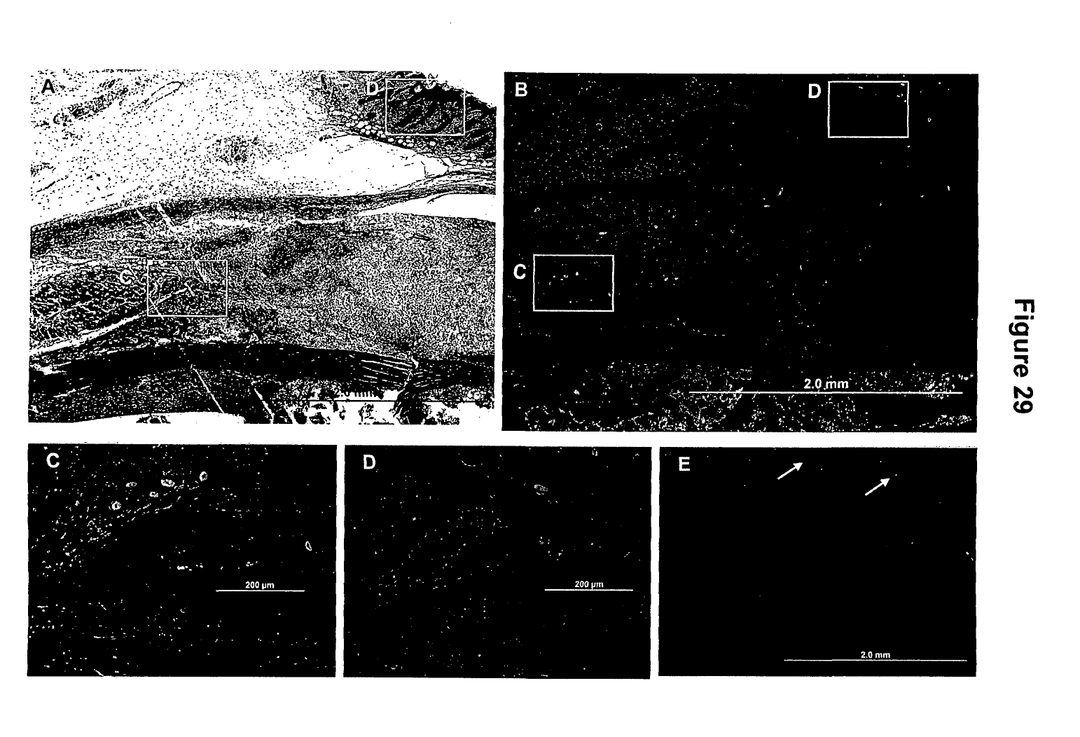

As muscle or fasciocutaneous tissue may be used to reconstruct the soft tissue envelope following open fracture in the clinical setting (Hallock, 2000, Naique et al., 2006, Yazar et al., 2006), we next evaluated the osteogenic potential of MDSC relative to cell populations derived from skin or adipose tissue. A comparison was also made with marrow-derived stromal cells, an alternative source of stromal cells readily availaBIe to the healing fracture. For this experiment, skin and fat were obtained from fasciocutaneous tissue, excised from off-cuts during vascularised flap reconstruction of open fractures as described for muscle previously. Cells isolated from fasciocutaneous tissue included skin fibroBIasts (SF) from the dermis (Toma et al., 2001) and fat-derived stromal cells (FDSC) from subcutaneous fat (Zuk et al., 2001). Marrow-derived stromal cells (MSC) were obtained from surgically cut tibiae. When cultured in osteogenic media for 4 weeks, both MDSC and MSC formed bone nodules, while the fat-derived stromal cells and the skin fibroBIasts did not (FIG. 24A). Additionally, alkaline phosphatase (ALP) expression measured at day 7, (see materials and methods) was significantly greater in MDSC and MSC than in either FDSC or SF. (FIG. 24B). Taken together, these data demonstrate that the osteogenic potential of MDSC in vitro is equivalent to that of MSC and exceeds that of fat-derived stromal cells and skin fibroBIasts.

Supernatants Derived from Fractured Bone Stimulate the Migration and Osteogenic Differentiation of MDSC In-Vitro.

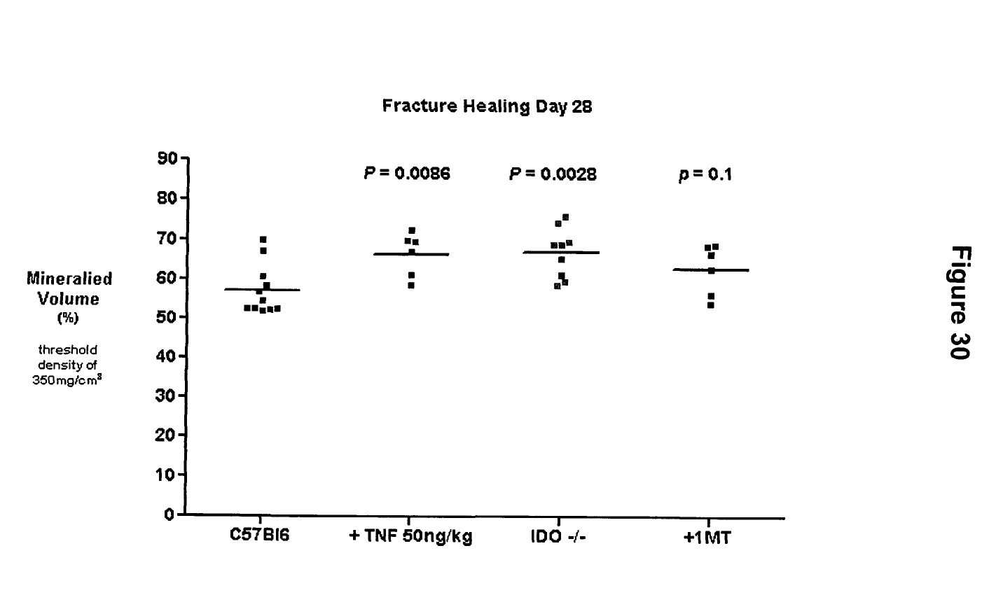

Factors that promote the migration and differentiation of the resident MDSC may provide a viaBIe therapeutic target as they would circumvent the requirement for the ex vivo expansion of stem cells for fracture repair therapy. To determine the effect of the fracture environment on MDSC in-vitro, supernatants were produced by culturing either fractured or surgically cut tibial fragments in serum free medium. Specimens were harvested during debridement of high-energy open tibial or ankle fractures. Fracture supernatants stimulated expression of ALP by MDSC at day 7, while supernatant from surgically cut bone did not (FIG. 25A). Migration of MDSC through an 8 μm transwell membrane varied between fracture supernatants, but all of the supernatants tested provided a migratory stimulus for MDSC significantly in excess of surgically cut bone supernatants (FIG. 25B). While all supernatants promoted MDSC proliferation in excess of that produced by human inactivated serum supernatant (HSS), as determined by cell counts after culture for 7 days, in this case there was no discerniBIe pattern between the fracture and surgically cut bone supernatants (FIG. 25C)

BMPs do not Account for the Osteogenic Stimulus of Fracture Supernatant BMPs, contained within cortical bone and produced by osteoprogenitor cells, are present within the fracture environment. As the response to BMPs in clinical trials has proved less efficacious than in animal models, where exogenous administration or viral transduction of BMPs (2, 4 and 7) accelerated fracture healing (Gerstenfeld et al., 2002, Musgrave et al., 2000, Peng et al., 2002, Shen et al., 2004), we sought to determine the influence of these pro-osteogenic factors on MDSC using our in vitro system. ELISAs specific for BMP2, BMP4, BMP7 (OP-1) and TGF-β estaBIished the presence of all four growth factors in a variety of fracture and control supernatants (TaBIe 2). There was, however, no association between the concentration in supernatant and the potency of the osteogenic stimulus, as measured by the ALP activity in culture, suggesting that BMPs were not responsiBIe for ALP expression by MDSCs (FIG. 25A).

TABLE 2
BMP concentrations in fracture and surgically cut bone supernatants.

| | | BMP2 | BMP4 | BMP7 | TGF-β |
|---|---|---|---|---|---|
| | Number | (pg/ml) | (pg/ml) | (pg/ml) | (pg/ml) |
| Fracture | 7 | 21.9 +/− 2.3 | 5.7 +/− | <5 | 1092 +/− |
| supernatant | | | 0.3 | | 6 |
| | 8 | 23.2 +/− 0.5 | 4.8 +/− | <5 | 486 +/− |
| | | | 0.7 | | 39 |
| | 9 | 17.8 +/− 2.3 | 8.2 +/− | <5 | 3644 +/− |
| | | | 1.3 | | 298 |
| | 10 | 16.5 +/− 2.1 | 5.3 +/− | <5 | 3693 +/− |
| | | | 1.1 | | 182 |
| | 11 | 18.8 +/− 2.8 | 8.9 +/− | <5 | 654 +/− |
| | | | 2.2 | | 49 |
| Atraumatically | 12 | 5.9 +/− 1.8 | 2.3 +/− | <5 | 1224 +/− |
| cut | | | 0.1 | | 42 |
| supernatant | 13 | 4.4 +/− 0.3 | 2.7 +/− | <5 | 313 +/− |
| | | | 0.1 | | 27 |
| | 14 | 17.3 +/− 12.6 | 3.5 +/− | <5 | 783 +/− |
| | | | 0.1 | | 65 |

TaBIe 2 Supernatants were obtained using fractured and surgically cut bone in serum free DMEM with 1% penicillin/streptomycin at 5 g of bone per ml. The supernatants were tested for the presence of BMP-2, BMP-4, BMP-7 and TGF-β using commercially availaBIe ELISA assays (all R+D systems, Abington, UK) according to the manufacturer's instructions. All assays were performed once, in triplicate, with values representing concentration ±1SD.

This observation was confirmed by culturing MDSC in fracture-derived supernatant with the addition of antibodies to BMP2/4, BMP7 and TGF-β. Antibody inhibition of the BMPs in supernatant did not suppress the osteogenic stimulus (FIG. 26A). MDSC were then cultured in human serum-containing media with the addition of human recombinant BMP2, BMP4, BMP7 and TGF-β. (As supra-physiological doses of BMPs were required to affect an osteogenic response in clinical trials (Friedlaender et al., 2001, Govender et al., 2002), it was anticipated that high concentrations would be required in our in vitro system and the range chosen reflected this). The addition of recombinant human BMPs, and TGF-β did not induce ALP expression (FIG. 26B). Moreover, BMPs 2, 4 and 7 did not induce cell proliferation, in excess of the media-only control (not shown).

TNF-α and IL-6 Promote the Osteogenic Differentiation of MDSC

It was reasoned that the principle difference between fracture fragments and surgically cut bone was in the environment from which they were harvested, and in particular the highly inflammatory nature of the milieu surrounding fracture fragments. Pro-inflammatory cytokines implicated in fracture repair include TNF-α, IL-1β and IL-6 (Dimitriou et al., 2005, Gerstenfeld et al., 2003a, Kon et al., 2001, Lehmann et al., 2005, Mountziaris and Mikos, 2008). Supernatants were evaluated using a 30-plex immuno-assay read using the Luminex xMAP® system. This system provides a means of rapidly assaying several cytokines simultaneously. This confirmed that both TNF-α and IL-1β were undetectaBIe to the limits of the assay, and that IL-6 was present in ng/ml concentrations (TaBIe 3). However, as it is likely that the concentrations encountered physiologically are of this magnitude (Gerstenfeld et al., 2003b) they were assumed to be present (Einhorn et al., 1995, Gerstenfeld et al., 2003b) and biologically active. Therefore, we sought to determine whether antibody-mediated inhibition of TNF-α, IL-1β and IL-6 in fracture supernatant inhibited the osteogenic stimulus. MDSC were cultured for 7 days in fracture supernatant with neutralising antibodies to either TNF-α, IL-1β or IL-6. ALP quantification revealed that AbTNF-α and AbIL-6 inhibited the osteogenic effect of fracture supernatant in a dose dependent manner, while AbIL-1β did not (FIG. 26C).

TABLE 3
Pro-inflammatory cytokine concentrations in fracture
and surgically cut bone supernatants.

| | | TNF-α | IL-1β | IL-6 |
|---|---|---|---|---|
| | Number | (pg/ml) | (pg/ml) | (pg/ml) |
| Fracture | 1 | <10 | <10 | 1841 +/− 92 |
| supernatant | 2 | <10 | <10 | 9522 +/− 476 |
| | 3 | <10 | <10 | 17671 +/− 283 |
| Atraumatically | 4 | <10 | <10 | 11414 +/− 171 |
| cut | 5 | <10 | <10 | 580 +/− 52 |
| supernatant | 6 | <10 | <10 | 764 +/− 82 |

TaBIe 3 Supernatants were obtained using fractured and surgically cut bone in serum free DMEM with 1% penicillin/streptomycin at 5 g of bone per ml. The supernatants were tested for the presence of TNF-α, IL-1β and IL-6 using a commercially availaBIe 30-plex assay (Invitrogen) with a Luminex xMAP® system (Luminex Corp., Austin Tex.) as per the manufacturer's instructions. All supernatants were tested once, in triplicate with the results given as pg/ml±1 SD.

The osteogenic effect of these cytokines on MDSC was then tested directly. RhTNF-α stimulated ALP expression by MDSC at a concentration of up to 1 ng/ml. Interestingly, ALP expression dropped sharply as the TNF-α concentration rose yet further, and was non-significantly less than the control at 100 ng/ml. By contrast, expression of ALP by IL-6 increased with the dose through the range tested. While a rise in ALP expression was demonstrated with IL-1β, peaking at 100 μg/ml, the response exhibited variability between donor cell populations, as evidenced by the relatively large error bars (FIG. 26D). MDSC were then cultured in human serum-containing media with rhTNF-α, rhIL-6 or rhIL-1β at the concentration optimized by FIG. 26D to determine whether ALP expression by MDSC cultured with cytokines was consistent with bone nodule formation. RhTNF-α in media stimulated vigorous nodule formation by MDSC in excess of that produced by rhIL-6. RhIL-1β did not stimulate bone nodule formation (FIG. 26E).

TNF-α and IL-6 Promote the Migration of MDSC

Next, the influence of TNF-α, IL-6 and IL-1β on the migration of MDSC in vitro was tested. Cell migration was tested in response to fracture supernatant with neutralising antibodies to either TNF-α, IL-10 or IL-6. Antibody inhibition of TNF-α in supernatant reduced the chemo attractant effect of the supernatant by around 50%. Antibody inhibition of IL-6 in supernatant reduced the chemo attractant effect of the supernatant by around 20%. Inhibition of IL-1β had no effect on cell migration (FIG. 27A). The influence of cytokines on MDSC migration was then tested directly. Both TNF-α and IL-6 were chemo attractant for MDSC within the dose-range tested. The optimal concentration of TNF-α for MDSC migration was 1 pg/ml, which was 1000-fold less than the optimal concentration for osteogenic differentiation. Similarly, the optimal concentration for IL-6, at 1 ng/ml, was also 1000-fold less than the concentration resulting in the highest ALP expression shown in FIG. 26D. IL-1β did not appear to promote MDSC migration at any of the concentrations tested (FIG. 27B)

It was hypothesized that as proliferation of MDSC cultured in supernatant was not fracture-dependent (see FIG. 25C), cytokines were not responsiBIe for the pattern of cellular proliferation seen. In order to test this hypothesis, proliferation of MDSC in human serum-containing media with rhTNF-α, rhIL-6 and rhIL-1β was tested using the CellTiter-Glo® luminescent cell viability assay was used (Promega Corp., Madison, Wis.).

This method assays the number of viaBIe cells in culture, by measuring adenosine triphosphate (ATP), where the luminescent signal is directly proportional to the number of ATP present. None of the cytokines resulted in cellular proliferation in excess of that observed using human serum-containing media alone. Proliferation was not dose-dependently-linked to the presence of pro-inflammatory cytokines TNF-α, IL-1β or IL-6 (FIG. 28).

MDSC Migrate to Fractured Bone In-Vivo.

Having found that MDSC can be stimulated to migrate and differentiate down an osteogenic lineage in response to inflammatory cytokines in vitro, we next sought evidence of MDSC migration and contribution to bone formation in response to the inflammatory fracture environment in vivo. Green fluorescent protein (eGFP) expressing MDSC were obtained from 8-week old heterozygous C57BI/6 female mice. Characterization of these cells revealed that they were of the mesenchymal stromal lineage (CD70 positive, CD90 positive, Ly6A/E negative) akin to human mesenchymal stromal cells (data not shown). The MDSC were injected into the pocket formed in the in vivo fracture model by the soft tissue dissection associated with circumferential periosteal stripping with or without mid-tibial osteotomy. The mice were recovered and were fully weight-bearing and freely mobile for a period of 7 days, when all mice were sacrificed and prepared for histological sectioning.

When stained with Masson's trichrome, the sections reveal a dense inflammatory milieu centrally. At the margins of the emerging callus is the formation of a cartilaginous tissue, with ossification of the extreme margin apparent (FIG. 29A). When observed under fluorescence microscopy, the histological sections of the fractured limb revealed the presence of eGFP expressing cells within the newly forming cartilaginous intermediary, and within the healing skin wound (FIG. 29B). The morphology of the eGFP cells within the cartilaginous intermediary is shown (FIG. 29C). The eGFP cells within the healing skin wound are located sub-epidermally (FIG. 29D). By contrast, eGFP expressing cells localised only to the skin wound as demonstrated in the histological sections of the periosteally stripped sham fractured limbs (FIG. 29E). These data suggest selective localization of eGFP-expressing MDSC.

Discussion

The in vivo recruitment and differentiation of osteoprogenitor cells for fracture healing remains incompletely understood. Although the importance of inflammation in early fracture healing has been estaBIished (17, 23) and recent work in experimental models has demonstrated that coverage with muscle improves healing time and union strength (32), an understanding of why an inflammatory environment combined with adequate access to muscle derived cells may be beneficial for fracture healing has not previously been described. This study has identified the potential of the adjacent skeletal muscle MDSCs to provide the osteoprogenitor cells needed to effect bony repair in periosteally stripped fractures, and may provide an explanation for the efficacy of muscle flap reconstruction on fracture healing time and strength, as the osteogenic potential of cells derived from muscle was shown to exceed that of the cell types availaBIe in fasciocutaneous flaps. It has demonstrated the importance of the pro-inflammatory cytokines TNF-α and IL-6 in MDSC in the recruitment and osteogenic differentiation of these cells. However, other progenitor cells are also likely to be involved in fracture healing, including mesenchymal stromal cells from the bone marrow.

We have shown that skeletal muscle lying posterior to periosteally stripped tibial fracture contains cells that are undergoing osteogenic differentiation, while the skin lying anteriorly does not. Furthermore, injected eGFP-labeled cells derived from skeletal muscle localized to the cartilaginous and osseous components of the fracture callus as well as the overlying skin wound, suggesting that these cells were directly involved in endochondral bone healing and perhaps also in cutaneous wound healing. Conversely, with the exception of the skin wound, there was no localization of eGFP MDSC in the sham fracture controls that underwent soft tissue dissection and periosteal stripping.

These data suggest that cells residing in muscle may be recruited by the fracture environment to act as a source of progenitor cells for new bone formation and that this recruitment is mediated by the early fracture environment itself. As we seek to translate experimental models into viaBIe therapeutic interventions, this approach may be desiraBIe for several reasons. Importantly, it relies on cells already present in tissue used to reconstruct the soft tissue envelope of open fractures. As our previous work would suggest, the abundance of these cells is sufficient to produce a union indistinguishaBIe from a closed fracture control (32). Therefore, it negates the need for the ex-vivo expansion and manipulation of cells from bone marrow or muscle and the separation of these cells into sub-populations for the purpose of implantation as an osteogenic population (9, 25). Moreover, our in vitro data are based on a heterogeneous cell population as present in native muscle, obviating the need, for sorting sub-populations of cells (25).

Using in vitro cell culture of human tissue, we have shown that the influence on migration and differentiation of MDSC by the early fracture environment is a function of trauma to the bone in situ and TNF-α mediates both cell recruitment and osteogenic differentiation. Using a simple, closed fracture healing model in dual receptor (p55^{-/-}/p75^{-/-}) knockout mice, the absence to TNF-α signalling was associated with delayed chondrogenic differentiation and maturation; delayed periosteal bone bridging, delayed callus resorption and delayed differentiation of the mesenchymal stromal cell infiltrate (39). The findings contradict those of Gilbert et al, who reported that TNF-α inhibited osteogenic differentiation of (rat) fetal calvarial cells and (murine) clonal osteoBIastic (MC3T3-E1-14) cells. These observations may have reflected properties of the non-human cells used (49, 50). Interestingly, as concentration of TNF-α increased, the pro-osteogenic effect rapidly declined. This observation was consistent with early in vivo studies demonstrating impaired fracture healing following high and repeated doses of TNF-α (51, 52). Moreover, in a co-culture system of osteoBIasts and marrow-derived cells, TNF-α decreased osteoclast number by >90% at 1 ng/ml, while osteoclastogenesis increased sharply at higher concentrations (53). These findings suggested that the equilibrium between bone resorption and deposition is critically influenced by the concentration of TNF-α. Our findings are also at variance of those of Lacey et al (2009), who found that TNF-α inhibited osteogenesis by mesenchymal stromal cells.

The osteogenic effect of TNF-α (and, to a lesser extend IL-6) may, in part, reflect down stream cellular expression of BMPs and other growth factors (48, 54, 55). Additionally, TNF-α modulates the expression of BMP receptors (56). Hence, the absence of BMP receptors on MDSC may have accounted for their lack of response to isolated recombinant human BMPs.

Antibody inhibition of TNF-α in supernatant reduced subsequent supernatant-mediated migration MDSC by around 50% at concentrations above 100 ng/ml. Moreover, recombinant human TNF-α promoted the migration of MDSC directly. In response to TNF-α migration may occur as a result of increased expression of cell adhesion molecules (57). However, in addition Ponte et al reported that TNF-α primed stromal cells for migration by stimulating expression of chemokine receptors CCR-2, CCR-3 and CCR-4 (58). Therefore TNF-α may be responsiBIe for recruitment of MDSC directly while also promoting the responsiveness of MDSC to other chemoattractant signals. This would account for the finding while TNF-α in media promoted cell migration, supernatant-mediated cell migration was only partially inhibited by antibody neutralization of TNF-α.

Antibody neutralization of IL-6 in supernatant similarly inhibited supernatant-mediated differentiation, although (unlike TNF-α) the osteogenic effect appeared to be directly proportional to concentration. Yet, while IL-6 promoted osteogenic differentiation of MDSC, it appeared to be less active than TNF-α in doing so. Moreover, while recombinant IL-6 promoted cell migration, the effect of neutralizing IL-6 on supernatant-mediated cell migration was modest. The apparent overlapping effects of TNF-α and IL-6 may arise as a result of IL-6 being a downstream factor in TNF-α-mediated cell recruitment and differentiation (59, 60).

IL-1β appeared to have a minor influence on fracture-mediated osteogenic differentiation of MDSC and no influence on fracture-mediated cell migration. Moreover, the influence of isolated IL-1β on osteogenic differentiation and migration was minimal. Specifically, IL-1β in isolation was not aBIe to promote bone nodule formation.

In summary, evidence was found for the presence of a population of muscle cells adjacent to fracture that, under influence of factors released by the fracture site, underwent osteogenic differentiation. Recruitment of these cells was demonstrated. TNF-α, present in purified samples from the fracture environment promoted the recruitment of osteogenic differentiation of these cells in vitro. These findings suggest that stromal cells, residing in skeletal muscle can be recruited as a source of osteoprogenitor cells for the purpose of fracture repair; a phenomenon that may be vital in circumstances such as high energy and open fractures at risk of delayed or non union. A translational strategy based on this concept might be developed as the clinical requirement for cover of open fractures by vascularised soft tissue such as muscle may, in addition provide the fracture with a reservoir of osteoprogenitor cells for fracture repair.

Materials and Methods

Obtaining a MDSC population

Skeletal muscle was harvested from C57/BI6 mice and from human subjects following surgical debridement and soft tissue reconstruction of lower limb trauma at Charing Cross Hospital (COREC No: 07/Q0411/30). All subsequent procedures were performed under sterile conditions in a laminar flow cabinet (Class II microbiological safety, Gelaire, Flow). Around 5 g of muscle was first washed briefly in Videne® solution (Adams Healthcare), and then rinsed three times in Hanks BSS (Invitrogen Corp.). The muscle was finely chopped using sterile scissors and placed in 20 ml of digestive medium (a filter-sterilized solution of 50 mg Collagenase II (Worthington Biochemical Corp.) and 100 mg Dispase (Invitrogen) dissolved in 20 ml warmed Hanks BSS in a 50 ml Falcon tube. The suspension was warmed to 37° C. and gently agitated for 30 minutes, centrifuged and resuspended in 12 ml culture medium (GIBCO® DMEM containing 50 ml (10%) GIBCO® FBS and 5 ml (1%) Penicillin/streptomycin (PAA Laboratories GmbH) and added to a 10 cm culture plate. Cell cultures were maintained in a humidified atmosphere of 5% CO₂ at 37° C. The culture media was changed at 24 hours and the plate assessed at day 3 for cell growth. Populations of skin fibroBIasts and fat-derived stromal cells were obtained using the same method.

Osteogenic Differentiation

Staining of MDSC for Alkaline Phosphatase

A population of plated MDSC was fixed with 1:1 100% acetone: 100% ethanol for 15 minutes, then rinsed 3 times. One SIGMAFAST™ tab (Sigma-Aldrich Corp.), was dissolved in 10 ml dH₂O and 0.5 ml added to each well. The plate was incubated at 37° C. for 30 minutes. Positive cells appeared dark purple under light microscopy. The images were taken at 40 times magnification, room temperature using an Olympus CKX41 microscope with a QICAM camera, (QIMAGING, GT vision LLC), and Q capture Pro® software.

Staining of MDSC for Bone Nodule Formation

1×10⁴ MDSC were added to wells of a 24-well plate in triplicate. The cells were cultured for 35-40 days in osteogenic media (DMEM with 10% fetal calf serum and 1% penicillin/streptomycin also containing 100 nM dexamethasone (SIGMA), 1 mM β-glycerol phosphate and 0.05 nM ascorbic acid), supernatant or media containing recombinant cytokines (with one media change per week) the cells were fixed with 2 ml 10% neutral buffered formalin at room temperature for 15 min. The plate was then washed twice with excess PBS. 1 ml filtered 40 mM alizarin red solution (pH 4.1) was added to each well and agitated at room temperature for 30 minutes. The excess dye was then aspirated and the plate washed four times in distilled water with gentle agitation. Finally, the plates were turned upside-down on paper to dry. Bone nodules appeared red under light microscopy. Images were taken at 20 times magnification and performed as above.

Adipogenic Differentiation

1×10⁴ human MDSC were added to wells of a 24 well plate (in triplicate). At 24 hrs the media was removed and replaced with 1 ml adipogenic induction media (h-insulin, L-glutamine, dexamethasone, indomethacin and 3-isobutyl 1-methylxanthine (IBMX) added to DMEM +10% FCS +1% P/S. from Lonza Group) At day 3 the media was removed and replaced with 1 ml adipogenic maintenance media (culture media containing h-insulin and L-glutamine (Lonza). Induction and maintenance media was used alternately for 21 days, with media changes every 3 days. The cells were then fixed in 10% formalin and stained with Oil red O as described below.

Oil-Red O Staining

Three parts filtered oil red O stock solution (containing 300 mg oil red O powder and 100 ml 99% isopropanol) were mixed with 2 parts deionised water and left at room temperature for 10 minutes. The fixed monolayer was rinsed in tap water. 1 ml 60% isopropanol was pipetted into each well and left for 5 minutes. The isopropanol was then poured off and 1 ml of the oil red O solution was added to each well. At 5 minutes this was also removed and the wells rinsed with tap water until the water ran clear. Images were taken at 20 times magnification and performed as above.

Chondrogenic Differentiation

1×10⁵ human MDSC were added to a 15 ml corning tube (in triplicate), and the tubes centrifuged at 1500 rpm for 5 minutes. The media was removed, leaving the cell pellet.

To 2 tubes, 1 ml chondrogenic media (ChondroDiff medium, Miltenyi Biotec) was added. To the third, standard (control) media was added. The medium was changed every 3 days. At day 35-40 days, the cell pellet was fixed by immersion in 10% neutral buffered formalin for at least 1 hour, then imbedded in paraffin prior to sectioning. The sections were stained using alcian BIue. The slides were viewed under 10 times magnification using an Olympus BX51 microscope (Olympus optical, Tokyo, Japan) at room temperature. Images were taken using an Olympus DP71 camera and DP controller and manager, Olympus software.

ALP Quantification Assay

1×10⁴ human MDSC were added to wells of a 96 well plate in triplicate. At 24 hrs the media was pipetted off and replaced by 200 μl test media. The media was changed every 3 days. At 7 days the media was removed and the cells lysed in 20 μl NP-40 lysis buffer. An ALP quantification assay (WAKO pure chemical Ltd.) was used. Calibration solution from the assay was serially diluted to make concentrations of 0.25, 0.125 and 0.0625 mmol/L. To empty wells, 20 μl of each calibration solution was added. To another, dH₂O was added. 100 μl reagent (1 taBIet per 5 ml buffer solution) was added to each well, and incubated at 37° C. for 20 minutes. 80 μl stop solution was then added to each well and the plate was read using an ELISA spectrometer at 405 nm wavelength. The concentration of ALP in the test wells was extrapolated from the standard curve.

Flow Cytometry

MDSC were divided into aliquots of around 1×10⁵ cells for each antibody used. Each aliquot was placed in a FACS tube. Briefly, the cells were suspended in 1 ml FACS Wash Buffer and placed on ice to cool. The fluorochrome-conjugated antibody was then added (volume as per manufacturer instructions, usually 10-20 μl). To further aliquots, isotype controls were added for each antibody and fluorochrome. The antibodies used were as follows: mouse anti-human PE isotype control, FITC isotype control and APC isotype control; PE conjugated mouse anti-human CD14, CD31, CD73, and CD90 (all BD Biosciences); FITC conjugated mouse anti-human CD34 (BD Biosciences) and CD105, CD146 and HLA-ABC (AbD Serotec); APC conjugated mouse anti-human CD45, CD106 and CD117 (all BD Biosciences). A further sample had no antibody added. The suspensions were then vortexed and incubated in darkness at 4° C. for 45 minutes. The cells were then washed twice with FACS Wash Buffer and resuspended in 1 ml FACS wash Buffer before being read immediately. The samples were read using a BD-LSR bench-top flow cytometer (BD Biosciences). Typically, staining was measured as an analysis of 10,000 events and expressed as a percentage of total cells. The data were analysed using Flowjo software for windows (Tree Star Inc.).

Supernatants

Human bone was obtained following debridement and surgical reconstruction of open tibial fractures or following amputation for un-reconstructaBIe severe open tibial fractures under the terms of the ethical approval granted by the combined office of research ethics committee (COREC No: 07/Q0411/30). Loose bone fragments or periosteally stripped bone fracture ends that were deemed to be non-viaBIe were excised using a surgical saw (Stryker) or bone nibBIers. These samples of bone were used to produce “fracture” supernatants. Where the limb was amputated, the tibia remote from the fracture site was sliced into segments using a surgical saw (Stryker). This bone was used to make surgically cut (control) supernatants.

Serum free media (DMEM+1% penicillin/streptomycin) was added to bone fragments or segments at 5 ml per gram of bone, and incubated for 12 hours. The supernatant was then filter-sterilised using a 0.2 μm filter (VWR) before being divided into 10 ml aliquots and stored at −80° C. prior to use.

MDSC Migration through a Transwell Membrane

500 μl serum free media was added to each well of a 24 well plate. 8 μm pore transwell membranes (VWR International Ltd) were then added to the wells. 1×10⁴ MDSC in serum free media were added to the upper chamber of each transwell membrane. At 12 hours the serum-free media was replaced with test or control media or supernatant and incubated at 37° C. for 36 hours. The upper chambers were then cleared of cells and cell debris using a cotton bud, in sweeping motions. The membrane was then washed and fixed using 10% neutral buffered formalin for 1 hour. After washing, the membrane was then added to a 1% crystal violet solution for 1 hour. After further washing, the membranes were viewed under light microscopy at 20 times magnification, using an Olympus CKX41 microscope with a QICAM camera, (QIMAGING, GT vision LLC). The number of cells present on the underside of the transwell membrane in any random field was counted 3 times and the mean calculated. Cell migration was calculated against the supernatant or human serum-containing (control) media and the number expressed as a percentage relative to the control media.

Murine Model

The mouse model was performed as described previously (Harry et al., 2008). Sham fracture included skin incision, soft tissue dissection, intramedullary reaming and insertion of pin, without surgical osteotomy. To each of the mice, 1×10⁵ eGFP expressing MDSC in 20 μl cold, sterile PBS were injected just deep to the superficial muscles of the posterior compartment, at the level of the shaft of tibia corresponding to the site of the surgical osteotomy, where performed. All animals were fed standard rodent chow and water ad libitum, and were housed (<six mice/cage) in sawdust-lined cages in an air-conditioned environment with 12 h light/dark cycles. All animal procedures were approved by the institutional ethics committee and the UK Home Office.

The mice were euthanized by means of cervical dislocation. The right lower limb was then harvested and placed immediately into 10% neutral buffered formalin. After 24 hours, the limb underwent radiographic imaging using the Faxitron MX-20 radiographic system (Faxitron X-Ray LLC, Lincolnshire, Ill.). If the fracture was excessively comminuted or if the intramedullary pin had extruded, the specimens were excluded from further analyses. Limbs with acceptaBIe fracture configurations then underwent decalcification for a period of 7 days in 10% formic acid. Using a scalpel, the intramedullary wire was used to guide division of the tibia in the sagittal plane, the wire was gently removed from the remaining sagittal section and this section embedded in paraffin wax. A microtome was used to cut 4 μm sections from the BIock. Sections were de-waxed with xylene and 99% absolute alcohol (industrial methylated spirit), then rehydrated in preparation for haematoxylin and eosin (H+E) staining. The slides were viewed under ×4 magnification using an Olympus BX51 microscope (Olympus optical, Tokyo, Japan) at room temperature. Images were taken using an Olympus DP71 camera and DP controller and manager, Olympus software, version 2.3.1.231. A fluorescent light source was used for the fluorescent images.

### REFERENCES

1. Bosse M J, MacKenzie E J, Kellam J F, et al. An analysis of outcomes of reconstruction or amputation after leg-threatening injuries. N Engl J Med. 2002; 347(24): 1924-31.

2. Castillo R C, MacKenzie E J, Wegener S T, et al. Prevalence of chronic pain seven years following limb threatening lower extremity trauma. Pain. 2006;124(3):321-9.

3. MacKenzie E J, Bosse M J, Kellam J F, et al. Early predictors of long-term work disability after major limb trauma. J Trauma. 2006;61(3):688-94.

4. McCarthy M L, MacKenzie E J, Edwin D, et al. Psychological distress associated with severe lower-limb injury. J Bone Joint Surg Am. 2003;85-A(9):1689-97.

5. Friedlaender G E, Perry C R, Cole J D, et al. Osteogenic protein-1 (bone morphogenetic protein-7) in the treatment of tibial nonunions. J Bone Joint Surg Am. 2001;83-A Suppl 1(Pt 2):S151-8.

6. Govender S, Csimma C, Genant H K, et al. Recombinant human bone morphogenetic protein-2 for treatment of open tibial fractures: a prospective, controlled, randomized study of four hundred and fifty patients. J Bone Joint Surg Am. 2002;84-A(12):2123-34.

7. Lane J M. BMPs: why are they not in everyday use? J Bone Joint Surg Am. 2001;83-A Suppl 1(Pt 2):S161-3.

8. Lieberman J R, Daluiski A, Einhorn T A. The role of growth factors in the repair of bone. Biology and clinical applications. J Bone Joint Surg Am. 2002;84-A(6): 1032-44.

9. Corsi K A, Schwarz E M, Mooney D J, et al. Regenerative medicine in orthopaedic surgery. J Orthop Res. 2007;25(10):1261-8.

10. Musgrave D S, Bosch P, Lee J Y, et al. Ex vivo gene therapy to produce bone using different cell types. Clin Orthop Relat Res. 2000(378):290-305.

11. Shen H C, Peng H, Usas A, et al. Structural and functional healing of critical-size segmental bone defects by transduced muscle-derived cells expressing BMP4. J Gene Med. 2004;6(9):984-91.

12. Wright V, Peng H, Usas A, et al. BMP4-expressing muscle-derived stem cells differentiate into osteogenic lineage and improve bone healing in immunocompetent mice. Mol Ther. 2002;6(2):169-78.

13. Baltzer A W, Lattermann C, Whalen J D, et al. Potential role of direct adenoviral gene transfer in enhancing fracture repair. Clin Orthop Relat Res. 2000(379 Suppl):S120-5.

14. Betz O B, Betz V M, Nazarian A, et al. Direct percutaneous gene delivery to enhance healing of segmental bone defects. J Bone Joint Surg Am. 2006; 88(2): 355-65.

15. Dragoo J L, Choi J Y, Lieberman J R, et al. Bone induction by BMP-2 transduced stem cells derived from human fat. J Orthop Res. 2003;21(4):622-9.

16. Huang X, Yang Y. Innate immune recognition of viruses and viral vectors. Hum Gene Ther. 2009;20(4):293-301.

17. Gerstenfeld L C, Cullinane D M, Barnes G L, et al. Fracture healing as a post-natal developmental process: molecular, spatial, and temporal aspects of its regulation. J Cell Biochem. 2003;88(5):873-84.

18. Shirley D, Marsh D, Jordan G, et al. Systemic recruitment of osteoBIastic cells in fracture healing. J Orthop Res. 2005;23(5):1013-21.

19. Kuznetsov S A, Mankani M H, Gronthos S, et al. Circulating skeletal stem cells. J Cell Biol. 2001;153(5):1133-40.

20. Eghbali-Fatourechi G Z, Lamsam J, Fraser D, et al. Circulating osteoBIast-lineage cells in humans. N Engl J Med. 2005;352(19):1959-66.

21. Urist MR. Bone: formation by autoinduction. Science. 1965;150(698):893-9.

22. McKibbin B. The biology of fracture healing in long bones. J Bone Joint Surg Br. 1978;60-B(2):150-62.

23. Einhorn TA. The cell and molecular biology of fracture healing. Clin Orthop Relat Res. 1998;355 Suppl:S7-21.

24. Lee J Y, Qu-Petersen Z, Cao B, et al. Clonal isolation of muscle-derived cells capaBIe of enhancing muscle regeneration and bone healing. J Cell Biol. 2000; 150(5):1085-100.

25. Qu-Petersen Z, Deasy B, Jankowski R, et al. Identification of a novel population of muscle stem cells in mice: potential for muscle regeneration. J Cell Biol. 2002;157(5):851-64.

26. Zuk P A, Zhu M, Mizuno H, et al. Multilineage cells from human adipose tissue: implications for cell-based therapies. Tissue Eng. 2001;7(2):211-28.

27. Toma J G, Akhavan M, Fernandes K J, et al. Isolation of multipotent adult stem cells from the dermis of mammalian skin. Nat Cell Biol. 2001;3(9):778-84.

28. Gopal S, Majumder S, Batchelor A G, et al. Fix and flap: the radical orthopaedic and plastic treatment of severe open fractures of the tibia. J Bone Joint Surg Br. 2000;82(7):959-66.

29. Yazar S, Lin C H, Lin Y T, et al. Outcome comparison between free muscle and free fasciocutaneous flaps for reconstruction of distal third and ankle traumatic open tibial fractures. Plast Reconstr Surg. 2006;117(7):2468-75; discussion 76-7.

30. Hallock G G. Utility of both muscle and fascia flaps in severe lower extremity trauma. J Trauma. 2000;48(5):913-7.

31. Naique S B, Pearse M, Nanchahal J. Management of severe open tibial fractures: the need for combined orthopaedic and plastic surgical treatment in specialist centres. J Bone Joint Surg Br. 2006;88(3):351-7.

32. Harry L E, Sandison A, Paleolog E M, et al. Comparison of the healing of open tibial fractures covered with either muscle or fasciocutaneous tissue in a murine model. J Orthop Res. 2008;26(9)1238-44.

33. Harry L E, Sandison A, Pearse M F, et al. Comparison of the Vascularity of Fasciocutaneous Tissue and Muscle for Coverage of Open Tibial Fractures. Plast Reconstr Surg. 2009;124(4):1211-9.

34. Dimitriou R, Tsiridis E, Giannoudis P V. Current concepts of molecular aspects of bone healing. Injury. 2005;36(12):1392-404.

35. Mountziaris P M, Mikos A G. Modulation of the inflammatory response for enhanced bone tissue regeneration. Tissue Eng Part B Rev. 2008;14(2):179-86.

36. Cho T J, Gerstenfeld L C, Einhorn T A. Differential temporal expression of members of the transforming growth factor beta superfamily during murine fracture healing. J Bone Miner Res. 2002;17(3):513-20.

37. Kon T, Cho T J, Aizawa T, et al. Expression of osteoprotegerin, receptor activator of NF-kappaB ligand (osteoprotegerin ligand) and related proinflammatory cytokines during fracture healing. J Bone Miner Res. 2001;16(6)1 004-14.

38. Lehmann W, Edgar C M, Wang K, et al. Tumor necrosis factor alpha (TNF-alpha) coordinately regulates the expression of specific matrix metalloproteinases (MMPS) and angiogenic factors during fracture healing. Bone. 2005;36(2):300-10.

39. Gerstenfeld LC, Cho T J, Kon T, et al. Impaired fracture healing in the absence of TNF-alpha signaling: the role of TNF-alpha in endochondral cartilage resorption. J Bone Miner Res. 2003;18(9):1584-92.

40. Heymann D, Rousselle A V. gp130 Cytokine family and bone cells. Cytokine. 2000; 12(10): 1455-68.

41. Yang X, Ricciardi B F, Hernandez-Soria A, et al. Callus mineralization and maturation are delayed during fracture healing in interleukin-6 knockout mice. Bone. 2007;41(6):928-36.

42. Yano S, Mentaverri R, Kanuparthi D, et al. Functional expression of beta-chemokine receptors in osteoBIasts: role of regulated upon activation, normal T cell expressed and secreted (RANTES) in osteoBIasts and regulation of its secretion by osteoBIasts and osteoclasts. Endocrinology. 2005;146(5):2324-35.

43. Jones S, Horwood N, Cope A, et al. The antiproliferative effect of mesenchymal stem cells is a fundamental property shared by all stromal cells. J Immunol. 2007; 179(5):2824-31.

44. Gerstenfeld L C, Cruceta J, Shea C M, et al. Chondrocytes provide morphogenic signals that selectively induce osteogenic differentiation of mesenchymal stem cells. J Bone Miner Res. 2002;17(2):221-30.

45. Peng H, Wright V, Usas A, et al. Synergistic enhancement of bone formation and healing by stem cell-expressed VEGF and bone morphogenetic protein-4. J Clin Invest. 2002;110(6):751-9.

46. Einhorn T A, Majeska R J, Rush E B, et al. The expression of cytokine activity by fracture callus. J Bone Miner Res. 1995;10(8):1272-81.

47. Kinnaird T, Stabile E, Burnett M S, et al. Local delivery of marrow-derived stromal cells augments collateral perfusion through paracrine mechanisms. Circulation. 2004; 109(12): 1543-9.

48. Rifas L. T-cell cytokine induction of BMP-2 regulates human mesenchymal stromal cell differentiation and mineralization. J Cell Biochem. 2006;98(4):706-14.

49. Gilbert L, He X, Farmer P, et al. Inhibition of osteoBIast differentiation by tumor necrosis factor-alpha. Endocrinology. 2000; 141(11): 3956-64.

50. Gilbert L C, Rubin J, Nanes M S. The p55 TNF receptor mediates TNF inhibition of osteoBIast differentiation independently of apoptosis. Am J Physiol Endocrinol Metab. 2005;288(5):E1011-8.

51. Hashimoto J, Yoshikawa H, Takaoka K, et al. Inhibitory effects of tumor necrosis factor alpha on fracture healing in rats. Bone. 1989;10(6):453-7.

52. Yoshikawa H, Hashimoto J, Masuhara K, et al. Inhibition by tumor necrosis factor of induction of ectopic bone formation by osteosarcoma-derived bone-inducing substance. Bone. 1988;9(6):391-6.

53. Balga R, Wetterwald A, Portenier J, et al. Tumor necrosis factor-alpha: alternative role as an inhibitor of osteoclast formation in vitro. Bone. 2006;39(2):325-35.

54. Fukui N, Ikeda Y, Ohnuki T, et al. Pro-inflammatory cytokine tumor necrosis factor-alpha induces bone morphogenetic protein-2 in chondrocytes via mRNA stabilization and transcriptional up-regulation. J Biol Chem. 2006;281(37):27229-41.

55. Yeh L C, Zavala M C, Lee J C. Osteogenic protein-1 and interleukin-6 with its soluBIe receptor synergistically stimulate rat osteoBIastic cell differentiation. J Cell Physiol. 2002;190(3):322-31.

56. Singhatanadgit W, Salih V, Olsen I. Bone morphogenetic protein receptors and bone morphogenetic protein signaling are controlled by tumor necrosis factor-alpha in human bone cells. Int J Biochem Cell Biol. 2006;38(10):1794-807.

57. von Andrian U H, Mackay C R. T-cell function and migration. Two sides of the same coin. N Engl J Med. 2000;343(14):1020-34.

58. Ponte A L, Marais E, Gallay N, et al. The in vitro migration capacity of human bone marrow mesenchymal stem cells: comparison of chemokine and growth factor chemotactic activities. Stem Cells. 2007;25(7):1737-45.

59. Franchimont N, Wertz S, Malaise M. Interleukin-6: An osteotropic factor influencing bone formation? Bone. 2005;37(5):601-6.

60. Kozawa O, Suzuki A, Kaida T, et al. Tumor necrosis factor-alpha autoregulates interleukin-6 synthesis via activation of protein kinase C. Function of sphingosine 1-phosphate and phosphatidylcholine-specific phospholipase C. J Biol Chem. 1997;272(40):25099-104.

1. A method of promoting bone formation in a patient at a site in need thereof, the method comprising the step of administering a pro-inflammatory compound to the site.

2.-4. (canceled)

5. The method of claim 1, wherein the site is a site of injury, a site of surgical intervention, a site requiring bone fusion or comprising damaged bone, eroded bone or bone defects.

6. (canceled)

7. The method of claim 5, wherein the surgical intervention is an osteotomy, a bone graft, an excision of bone from a donor site for a bone graft, the insertion of an implant into, around and/or adjacent to a bone or the fixing an implant to a bone.

8. The method of claim 1, wherein the promotion of bone formation aids in repairing bone, accelerating bone formation, increasing cortical bone volume, increasing cortical bone mineral content and/or increasing bone mineral density at the site, increasing mineralised volume of the healing bone, the mineralised bone volume fraction and tissue mineral density.

9. The method of claim 7, wherein the implant is selected from, the group comprising a joint replacement, a dental implant, a pin, a plate, a screw, an intramedullary device and/or an intraosseous device.

10. The method of claim 9, wherein adherence of the implant to the bone is strengthened in comparison with adherence of an implant to bone in the absence of the method of claim 9.

11.-13. (canceled)

14. The method of claim 1, wherein the patient has compromised bone due to metabolic bone disorders hereditary bone conditions, osteoporosis, infection, malignant or benign tumours affecting bone, bone affected by chemotherapy, radiotherapy and/or disuse.

15.-16. (canceled)

17. The method of claim, wherein the promotion of bone formation augments and/or accelerates bone formation during distraction lengthening.

18.-19. (canceled)

20. The method of claim 1, wherein the pro-inflammatory compound is for administration immediately following injury or surgery.

21. The method of claim 1, wherein the pro-inflammatory compound is for administration between one hour and one year after the injury or surgical intervention.

22. The method of claim 1, wherein the pro-inflammatory compound is for administration at the time of surgical intervention or injury.

23. A kit of parts comprising a surgical implant in combination with a pro-inflammatory compound.

24. (canceled)

25. The kit of claim 23, wherein the pro-inflammatory compound is dispersed within the cement.

26. The kit of claim 23, wherein the surgical implant and/or cement is coated with the pro-inflammatory compound.

27.-28. (canceled)

29. The kit of claim 23, wherein the pro-inflammatory compound is, or is used in combination with, an inhibitor of indoleamine 2, 3, dioxygenase 1 (IDO).

30.-31. (canceled)

32. The kit of claim 29, wherein the inhibitor of IDO is a compound that inhibits the activity of IDO by acting upstream or downstream of IDO in inflammation.

33.-36. (canceled)

37. The of claim 23, wherein the pro-inflammatory compound is a pro-inflammatory cytokine

38. The kit of claim 23, wherein the pro-inflammatory cytokine is TNFα.

39.-48. (canceled)

49. The method of claim 1, wherein the pro-inflammatory compound is, or is used in combination with, an inhibitor of indoleamine 2, 3, dioxygenase 1 (IDO).

50. The method of claim 49, wherein the inhibitor of IDO is a compound that inhibits the activity of IDO by acting upstream or downstream of IDO in inflammation.

51. The method of claim 1, wherein the pro-inflammatory compound is a pro-inflammatory cytokine.

52. The method of claim 1, wherein the pro-inflammatory cytokine is TNFα.
